(19) United States
(12) Patent Application Publication
(10) Pub. No.: US 2026/0091118 A1
(43) Pub. Date: Apr. 2, 2026

(54) Methods for Preparation and Use of BacPROTACs for Targeted Protein Degradation in Mycobacteria and Other Bacteria

(71) Applicant: Washington University, St. Louis, MO (US)

(72) Inventors: Timothy Wencewicz, St. Louis, MO (US); Emily Williford, St. Louis, MO (US); Helen Blaine, St. Louis, MO (US); Christina Stallings, St. Louis, MO (US)

(73) Assignee: Washington University, St. Louis, MO (US)

(21) Appl. No.: 19/312,129

(22) Filed: Aug. 27, 2025

Related U.S. Application Data
(60) Provisional application No. 63/687,679, filed on Aug. 27, 2024.

(51) Int. Cl. A61K 47/55; A61K 47/54; A61K 47/60; A61P 31/04

(52) U.S. Cl. CPC A61K 47/552; A61K 47/543; A61K 47/60; A61P 31/04

(57) ABSTRACT

Among the various aspects of the present disclosure is the provision of bivalent agents for targeted protein degradation. Also disclosed are compositions including the bivalent agents and methods of use, including methods of reducing antibiotic resistance associated with an antibiotic-resistant bacterial infection and methods of treating an antibiotic-resistant bacterial infection.

### CROSS-REFERENCE TO RELATED APPLICATIONS

This application claims the benefit of priority to U.S. Provisional Application Ser. No. 63/687,679 filed on Aug. 27, 2024, which is incorporated herein by reference in its entirety.

### STATEMENT REGARDING FEDERALLY SPONSORED RESEARCH OR DEVELOPMENT

This invention was made with government support under AI123394 awarded by the National Institutes of Health (NIH) and DGE2139839 awarded by the National Science Foundation (NSF). The government has certain rights in the invention.

### MATERIAL INCORPORATED-BY-REFERENCE

Not applicable.

### FIELD OF THE INVENTION

The present disclosure generally relates to compositions of tetracycline destructase inhibitors and methods of use thereof.

### BACKGROUND OF THE INVENTION

Tetracycline (TC) antibiotics are a family of type-II polyketides originally isolated from Streptomyces aureofaciens. TCs have been in clinical use for over 70 years as broad-spectrum antibiotics and continue to be used as frontline agents for treating a variety of infections caused by Gram-positive and Gram-negative bacteria. Until recently, it was thought that clinical TC resistance occurred primarily through the expression of efflux pumps and ribosome protection proteins. These resistance mechanisms have been largely overcome in the clinic by the development of last-generation TCs known as glycylcyclines including the FDA-approved drugs tigecycline, eravacycline, and omadacycline. Unfortunately, all known TC antibiotics are susceptible to an emerging third route of clinical resistance: enzymatic inactivation by tetracycline destructase (TDase) enzymes. TDases are members of the class A flavin monooxygenase (FMO) enzyme family. TDases are FAD-dependent and use an NADPH/02-coupled redox cycle to catalyze the inactivation of TC antibiotics.

Tetracyclines are essential antibiotics for treating a wide range of clinical infections but are threatened by an emerging resistance mechanism—enzymatic inactivation by tetracycline destructases (TDases). Recently developed inhibitors of this class of enzymes have shown that anhydrotetracycline (aTC) and aTC analogs are able to rescue tetracycline activity in vitro and in E. coli expressing Type I (TetX7) and Type II (Tet50) TDase enzymes.

Targeted protein degradation has largely been used as a tool in anti-cancer therapies to target cancer causing proteins to the proteasome, but until recently there have been few advances in the field of bacterial targeted protein degradation. Recently, there have been two published bacterial proteolysis targeting chimeras (BacPROTACs) that are effective in Mycobacteria. These bifunctional molecules utilize a phosphorylated guanidine, which mimics a tagged protein from a translation stalling event, or a cyclic peptide warhead, which works by dysregulating the protease, resulting in nonspecific degradation of proteins. Both warheads target and activate the ClpC1P1P2 proteasome complex present in Mycobacteria.

### SUMMARY OF THE INVENTION

Among the various aspects of the present disclosure is the provision of compositions of bifunctional inhibitors of tetracycline inactivating enzymes and methods of use thereof.

Briefly, therefore, the present disclosure is directed to compositions containing bifunctional inhibitors of tetracycline destructase (TDase) and their use in the treatment of bacterial infections.

In one aspect, a composition for degradation of a targeted enzyme is provided that includes a bivalent agent. The bivalent agent includes a targeted enzyme binding moiety configured to selectively bind to the targeted enzyme; a proteasome targeting moiety configured to selectively bind to a peptidase component of a proteosome; and a linker moiety, wherein the targeted protein binding moiety and protein degradation moiety are covalently linked at opposite ends of the linker moiety. In some aspects, the proteasome targeting moiety comprises a peptide configured to selectively bind to a ClpP1P2 protease complex of a proteosome. In some aspects, the proteasome targeting moiety is selected from a cyclic acyl depsipeptide (ADEP) or portion thereof, and (S,E)-(3,5 difluorophenyl-2-)hept-2-eneamido)propanoic acid (DFP). In some aspects, the targeted enzyme binding moiety comprises a tetracycline destructase (TDase) inhibitor selected from anhydrotetracycline (aTC) or an aTC analog. In some aspects, the linker molecule comprises a polyethylene glycol (PEG) linker. In some aspects, the PEG linker comprises a linear series of PEGs ranging from about three PEGS (PEG3) to about eight PEGS (PEG8). In some aspects, the bivalent agent comprises the aTC targeted enzyme binding moiety and the DFP proteasome targeting moiety covalently bound to opposite ends of the PEG linker. In some aspects, the bivalent agent comprises a chemical structure according to Formula (I) below:

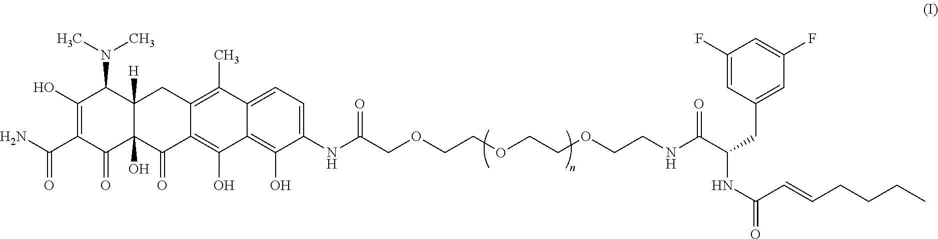

wherein n ranges from 3 to 8. In some aspects, the bivalent agent is selected from aTC-PEG₃-DFP (PEG₃-5), aTC-PEG₃-DFP (PEG₆-5), and aTC-PEG₈-DFP (PEG₈-5) comprising the chemical structures illustrated below:

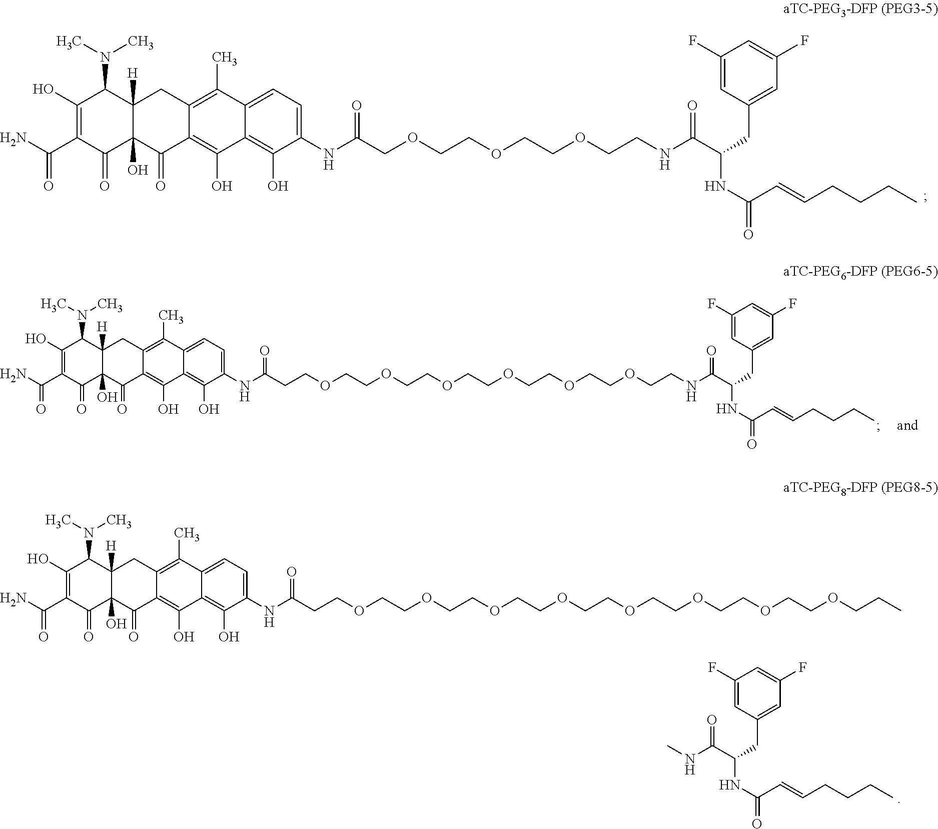

In some aspects, n is 6.

In another aspect, a method of reducing antibiotic resistance associated with an antibiotic-resistant bacterial infection in a subject is disclosed. The method includes administering a therapeutically effective amount of a composition comprising a bivalent agent to the subject, wherein the bivalent agent includes a targeted enzyme binding moiety comprising anhydrotetracycline (aTC) and a proteasome targeting moiety comprising (S,E)-(3,5 difluorophenyl-2-)hept-2-eneamido)propanoic acid (DFP) attached at opposite ends of polyethylene glycol (PEG) linker. In some aspects, the bivalent agent comprises a chemical structure according to Formula (I) below:

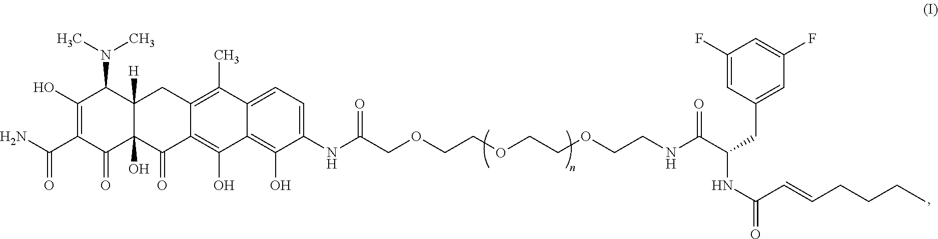

wherein n ranges from 3 to 8. In some aspects, the antibiotic-resistant bacterial infection is caused by a bacterial pathogen selected from Escherichia coli, Acinetobacter baumannii, Pseudomonas aeruginosa, Legionella longbeacha, and Mycobacteria abscessus. In some aspects, the composition administered to the subject comprises the bivalent agent at a concentration ranging from about 100 μM to about 1 mM.

In an additional aspect, a method of treating an antibiotic-resistant bacterial infection in a subject, the method comprising administering a therapeutically effective amount of a composition comprising a bivalent agent to the subject, wherein the bivalent agent comprises a targeted enzyme binding moiety comprising anhydrotetracycline (aTC) and a proteasome targeting moiety comprising (S,E)-(3,5 difluorophenyl-2-)hept-2-eneamido)propanoic acid (DFP) attached at opposite ends of a, and a the aTC targeted enzyme binding moiety and the DFP proteasome targeting moiety covalently bound to opposite ends of a polyethylene glycol (PEG) linker. In some aspects, the bivalent agent comprises a chemical structure according to Formula (I) below

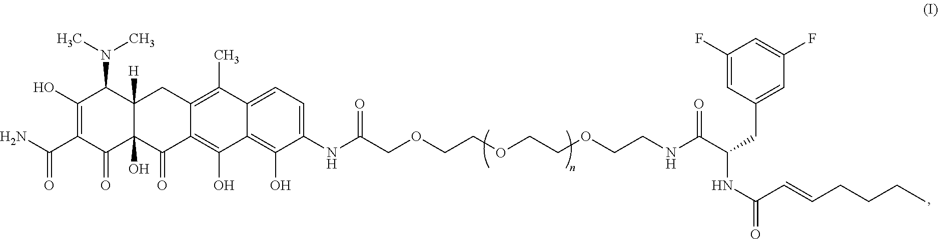

wherein n ranges from 3 to 8. In some aspects, the antibiotic-resistant bacterial infection is caused by a bacterial pathogen selected from Escherichia coli, Acinetobacter baumannii, Pseudomonas aeruginosa, Legionella longbeacha, and Mycobacteria abscessus. In some aspects, the composition administered to the subject comprises the bivalent agent at a concentration ranging from about 100 μM to about 1 mM. In some aspects, the composition administered to the subject further comprises a tetracycline antibiotic. In some aspects, the tetracycline antibiotic of the composition is administered to the subject at a concentration of about 8 μM.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a graphical representation of targeted protein degradation in Mycobacteria using current proteolysis targeting chimeras (PROTAC) which bind ClpP1 ligands and PROTACs which bind ClpC1 ligands (top). On the bottom are tetracycline destructase (TDase) inhibitor (“Target’) binding a proteasome complex. A known inhibitor of TDase, aTC, is conjugated to a ClpP1 targeting ligand or a ClpC1 targeting ligand to induce proximity to the ClpP1P2 (bottom; right) or ClpP1P2C1 (bottom; left) proteasome complexes, respectively, and promote proteolysis of the target protein.

FIG. 2 is a schematic of the synthesis of bivalent TDase degraders acting of ClpP1P2.

FIG. 3 is a schematic of aTC-PEGₙ-DFP (top) and a set of western blot images for RpoB (control) and TetX-HA in Mycobacteria smegmatis treated with variable concentrations of bivalent protein degraders aTC-PEGₙ-DFP (PEG₃-5, PEG₆-5, PEG₈-5).

FIG. 4 is an image of the aTC-PEG₆-DFP compound (top), an SDS-PAGE gel (middle), and a schematic (bottom). The ClpTAC compound PEG₆-5 promotes the in vitro degradation of TetX by ClpP1P2 at 37° C. Addition of ClpC1 inhibits ClpTAC mediated TetX degradation. SDS-PAGE gels show bands for TetX (˜45 kDa) after 3 hrs incubation at 37° C.

FIG. 5 is a set of images of showing the minimum inhibitory concentration (MIC) of Mycobacterium smegmatis expressing TetX-HA MICs of TC, aTC, PEG₆-4, and PEG₆-5 alone.

FIG. 6A is an image of a 96-well plate showing the fractional inhibitory concentration index (FICI) of PEG₆-5 and TC show PEG₆-5 dose dependent rescue of TC at 2 μg/mL TC in M. smegmatis expressing TetX-HA.

FIG. 6B is an image of a 96-well plate showing MIC of PEG₆-4 and PEG₆-5 show ClpTAC rescue of TC activity between 250 μM to 500 μM in M. smegmatis expressing TetX-HA.

FIG. 7A is a chart showing growth inhibitory activity of aTC, ClpTAC PEG₆-5, and PEG₆-4 against Mycobacterium abscessus L948 in combination with Tet.

FIG. 7B is a graph showing the growth inhibitory activity of aTC. The percent inhibition of growth in checkerboard assays of test compounds with Tet in combination at various concentrations. Comparison of the percent inhibition of growth for aTC and Tet at the specified concentrations.

FIG. 7C is a graph showing the growth inhibitory activity of ClpTAC PEG₆-5. The percent inhibition of growth in checkerboard assays of test compounds with Tet in combination at various concentrations. Comparison of the percent inhibition of growth for ClpTAC PEG₆-5 and Tet at the specified concentrations.

FIG. 7D is a graph showing the growth inhibitory activity of ClpTAC PEG₆-4. The percent inhibition of growth in checkerboard assays of test compounds with Tet in combination at various concentrations. Comparison of the percent inhibition of growth for ClpTAC PEG₆-4 and Tet at the specified concentrations.

FIG. 8 is a schematic showing the synthesis of ClpC1-targeting bacterial PROTAC.

FIG. 9 is a schematic and corresponding graphical representation of a PROTAC synthesis and design. The schematics show the pupylation pathway (top), the proposed pupylation of Tet(X) (middle), and the design of PROTAC (bottom). The graphical representation is a model of PROTAC in Tet(X).

FIG. 10 is a schematic showing the synthesis of an electrophilic “scout” molecule for PafA ligand discovery.

FIG. 11 is a set of graphs showing ClpP1P2 protease activity at room temperature (top) and at 4° C. (bottom) using AMC fluorescence.

FIG. 12 is an image of an SDS-PAGE gel showing bands for TetX after 3 hours incubation at 37° C. with the indicated molecules.

FIG. 13 is a graph showing PEG₆-5 inhibition of TetX.

FIG. 14 is a set of images of SDS-PAGE gels showing the BacPROTAC compound PEG₆-5 promotes the in vitro degradation of TetX by ClpP1P2C1 at 37° C.

FIG. 15 is a set of images showing the Mycobacterium smegmatis minimum inhibitory concentration (MIC) of PROTAC in combination with TC. Pmv261+TetX (top) and Pmsg430+TetX (bottom).

FIG. 16 is a structure (top) and set of graphs (bottom) of LC-MS analysis with stacked plots of optical absorbance (260 nm; top), total ion counts (middle), and extracted ion counts (m/z 547; bottom). The x-axis for all three chromatograms is retention time (min). The y-axis for the top trace is milli absorbance units (mAU). The y-axes for the middle and bottom traces are ion counts.

FIG. 17 is a structure (top) and set of graphs (bottom) of LC-MS analysis with stacked plots of optical absorbance (260 nm; top), total ion counts (middle), and extracted ion counts (m/z 391; bottom). The x-axis for all three chromatograms is retention time (min). The y-axis for the top trace is milli absorbance units (mAU). The y-axes for the middle and bottom traces are ion counts.

FIG. 18 is a structure (top) and set of graphs (bottom) of LC-MS analysis with stacked plots of optical absorbance (260 nm; top), total ion counts (middle), and extracted ion counts (m/z 501; bottom). The x-axis for all three chromatograms is retention time (min). The y-axis for the top trace is milli absorbance units (mAU). The y-axes for the middle and bottom traces are ion counts.

FIG. 19 is a 1H-NMR graph.

FIG. 20 is a 1H-NMR graph.

FIG. 21 is a structure (top) and a 1H-NMR graph (bottom).

FIG. 22 is a 13C-NMR graph.

FIG. 23 is a COSY graph.

FIG. 24 is an HSQC graph.

FIG. 25 is an HMBC graph.

FIG. 26 is a TOCSY graph.

FIG. 27 is a structure (top) and set of graphs (bottom) of LC-MS analysis with stacked plots of optical absorbance (260 nm; top), total ion counts (middle), and extracted ion counts (m/z 694; bottom). The x-axis for all three chromatograms is retention time (min). The y-axis for the top trace is milli absorbance units (mAU). The y-axes for the middle and bottom traces are ion counts.

FIG. 28 is a structure (top) and set of graphs (bottom) of LC-MS analysis with stacked plots of optical absorbance (260 nm; top), total ion counts (middle), and extracted ion counts (m/z 538; bottom). The x-axis for all three chromatograms is retention time (min). The y-axis for the top trace is milli absorbance units (mAU). The y-axes for the middle and bottom traces are ion counts.

FIG. 29 is a structure (top) and set of graphs (bottom) of LC-MS analysis with stacked plots of optical absorbance (260 nm; top), total ion counts (middle), and extracted ion counts (m/z 648; bottom). The x-axis for all three chromatograms is retention time (min). The y-axis for the top trace is milli absorbance units (mAU). The y-axes for the middle and bottom traces are ion counts.

FIG. 30 is a 1H-NMR graph.

FIG. 31 is a 13C-NMR graph.

FIG. 32 is a structure (top) and set of graphs (bottom) of LC-MS analysis with stacked plots of optical absorbance (260 nm; top), total ion counts (middle), and extracted ion counts (m/z 536; bottom). The x-axis for all three chromatograms is retention time (min). The y-axis for the top trace is milli absorbance units (mAU). The y-axes for the middle and bottom traces are ion counts.

FIG. 33 is a 1H-NMR graph.

FIG. 34 is a 13C-NMR graph.

FIG. 35 is a structure (top) and set of graphs (bottom) of LC-MS analysis with stacked plots of optical absorbance (260 nm; top), total ion counts (middle), and extracted ion counts (m/z 782; bottom). The x-axis for all three chromatograms is retention time (min). The y-axis for the top trace is milli absorbance units (mAU). The y-axes for the middle and bottom traces are ion counts.

FIG. 36 is a structure (top) and set of graphs (bottom) of LC-MS analysis with stacked plots of optical absorbance (260 nm; top), total ion counts (middle), and extracted ion counts (m/z 626; bottom). The x-axis for all three chromatograms is retention time (min). The y-axis for the top trace is milli absorbance units (mAU). The y-axes for the middle and bottom traces are ion counts.

FIG. 37 is a structure (top) and set of graphs (bottom) of LC-MS analysis with stacked plots of optical absorbance (260 nm; top), total ion counts (middle), and extracted ion counts (m/z 648; bottom). The x-axis for all three chromatograms is retention time (min). The y-axis for the top trace is milli absorbance units (mAU). The y-axes for the middle and bottom traces are ion counts.

FIG. 38 is a structure (top) and set of graphs (bottom) of LC-MS analysis with stacked plots of optical absorbance (260 nm; top), total ion counts (middle), and extracted ion counts (m/z 580; bottom). The x-axis for all three chromatograms is retention time (min). The y-axis for the top trace is milli absorbance units (mAU). The y-axes for the middle and bottom traces are ion counts.

FIG. 39 is a 1H-NMR graph.

FIG. 40 is a 13C-NMR graph.

### DETAILED DESCRIPTION OF THE INVENTION

The present disclosure is based, at least in part, on the discovery that anhydrotetracycline (aTC) and aTC analogs are able to rescue Type I (TetX7) and Type II (Tet50) tetracycline activity.

One aspect of the present disclosure provides the targeted degradation of TDases by the ClpP1P2 protease component of the proteasome. A bivalent BacPROTAC molecule capable of binding both ClpP1P2 and TDase induces proximity and promotes the targeted proteolysis of TDase. The PROTAC-like molecule is composed of the known TDase inhibitor aTC joined covalently to a ClpP1P2-targeting small molecule, (S,E)-(3,5-difluorophenyl)-2-(hept-2-eneamido)propanoic acid (DFP), through a polyethylene glycol (PEG) linker of variable length.

In one aspect, the ClpP1P2-targeting ligand could also be the complete cyclic acyl depsipeptides (ADEPs). In one aspect, the ClpC1 chaperone component could be targeted using a cyclomarin derivative conjugated via PEG linkage to aTC.

### Tetracycline Destructase (TDase) Modulation Agents

As described herein, TDase expression has been implicated in various diseases, disorders, and conditions, including tetracycline-resistant bacterial infections. As such, modulation of TDase (e.g., modulation of its ability to degrade tetracycline) in concert with administration of a tetracycline compound can be used for treatment of such conditions. A TDase modulation agent can modulate TDase response or induce or inhibit TDase. TDase modulation can comprise modulating the expression of TDase in, on, and outside cells, modulating the quantity of cells that express TDase, or modulating the quality of the TDase-expressing cells.

TDase modulation agents can be any composition or method that can modulate TDase expression on cells (e.g., by binding TDase). For example, a TDase modulation agent can be an activator, an inhibitor, an agonist, or an antagonist. As another example, TDase modulation can be the result of gene editing. TDase modulation can be targeting TDase for proteasomal degradation.

A TDase modulation agent can be a TDase antibody (e.g., a monoclonal antibody to TDase).

A TDase modulating agent can be an agent that induces or inhibits progenitor cell differentiation into TDase expressing cells. For example, TDase small molecule inhibitors can be used to block TDase.

TDase Signal Reduction, Elimination, or Inhibition by Small Molecule Inhibitors, shRNA, siRNA, or ASOs

As described herein, a TDase modulation agent can be used for use in tetracycline-resistant bacterial infection therapy. A TDase modulation agent can be used to reduce/eliminate or enhance/increase TDase signals. For example, a TDase modulation agent can be a small molecule inhibitor of TDase. As another example, a TDase modulation agent can be a short hairpin RNA (shRNA). As another example, a TDase modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### TDase Inhibiting Agent

One aspect of the present disclosure provides for targeting of TDase, its substrate, or its downstream signaling. The present disclosure provides methods of treating or preventing bacterial infections based on the discovery that by appending a nicotinamide isostere to the C9-position of the aTC D-ring, bisubstrate tetracycline reductase (TDase) inhibitors are generated. The bisubstrate inhibitors can have extended interactions with TDases by spanning both the TC and presumed NADPH binding pockets. This can simultaneously block TC binding and reduction of FAD by NADPH while ‘locking’ TDases in an unproductive FAD ‘out’ conformation.

As described herein, inhibitors of TDase (e.g., antibodies, fusion proteins, small molecules) can reduce or prevent TC-resistant bacterial infections. A TDase inhibiting agent can be any agent that can inhibit TDase, downregulate TDase, or knockdown TDase.

As an example, a TDase inhibiting agent can inhibit TDase signaling.

For example, the TDase inhibiting agent can be an anti-TDase antibody. Furthermore, the anti-TDase antibody can be a murine antibody, a humanized murine antibody, or a human antibody.

As another example, the TDase inhibiting agent can be an anti-TDase antibody, wherein the anti-TDase antibody prevents binding of TDase to its receptor or prevents activation of TDase and downstream signaling.

As another example, the TDase inhibiting agent can be a fusion protein. For example, the fusion protein can be a decoy receptor for TDase. Furthermore, the fusion protein can comprise a mouse or human Fc antibody domain fused to the ectodomain of TDase.

As another example, a TDase inhibiting agent can be any of the small molecules described in the present disclosure, which have been shown to be a potent and specific inhibitor of TDase signaling.

As another example, a TDase inhibiting agent can be anhydrotetracycline (aTC) or an aTC analog.

As another example, a TDase inhibiting agent can be an inhibitory protein that antagonizes TDase. For example, the TDase inhibiting agent can be a viral protein, which has been shown to antagonize TDase.

As another example, a TDase inhibiting agent can be a short hairpin RNA (shRNA) or a short interfering RNA (siRNA) targeting TDase.

As another example, a TDase inhibiting agent can be an sgRNA targeting TDase.

Methods for preparing a TDase inhibiting agent (e.g., an agent capable of inhibiting TDase signaling) can comprise the construction of a protein/Ab scaffold containing the natural TDase receptor as a TDase neutralizing agent; developing inhibitors of the TDase substrate “down-stream”; or developing inhibitors of the TDase production “up-stream”.

Inhibiting TDase can be performed by genetically modifying TDase in a subject or genetically modifying a subject to reduce or prevent expression of the TDase gene, such as through the use of CRISPR-Cas9 or analogous technologies, wherein, such modification reduces or prevents a bacterial infection.

### Chemical Agent

Examples of tetracycline antibiotic rescue agents are described herein, including tetracycline destructase inhibitors. Agents (and compositions comprising one or more agents) can comprise one or more of a compound comprising anhydrotetracycline (aTC), a polyethylene glycol (PEG) linker, and (S,E)-(3,5-difluorophenyl)-2-(hept-2-eneamido)propanoic acid (DFP) (aTC-PEGₙ-DFP), including compounds of the formula

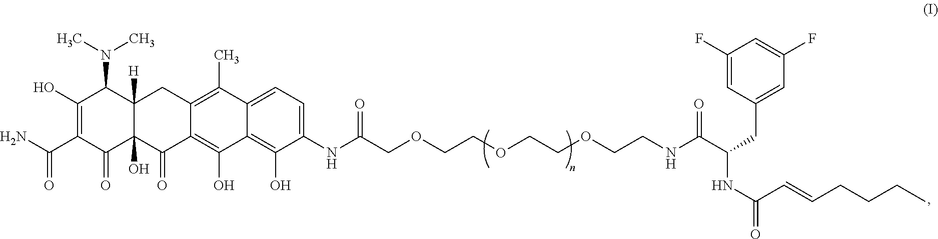

or pharmaceutically acceptable salts thereof. In exemplary embodiments, PEGₙ may comprise PEG₃, PEG₅, and PEG₈.

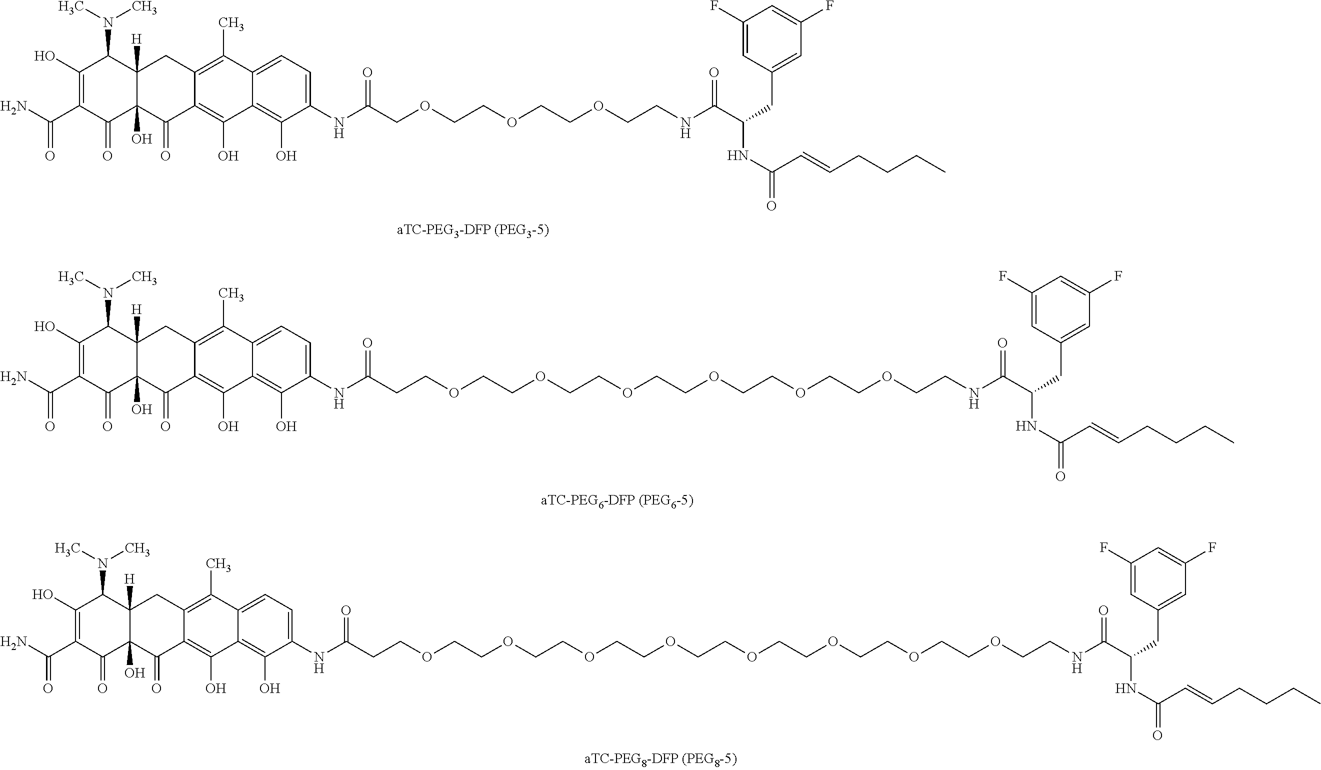 [SMILES: CCCC/C=C/C(=O)N[C@@H](Cc1cc(F)cc(F)c1)C(=O)NCCOCCOCCOCC(=O)Nc1ccc2c(C)c3c(c(O)c2c1O)C(=O)[C@]1(O)C(=O)C(C(N)=O)=C(O)[C@@H](N(C)C)[C@@H]1C3 ; CCCC/C=C/C(=O)N[C@@H](Cc1cc(F)cc(F)c1)C(=O)NCCOCCOCCOCCOCCOCCOCCC(=O)Nc1ccc2c(C)c3c(c(O)c2c1O)C(=O)[C@]1(O)C(=O)C(C(N)=O)=C(O)[C@@H](N(C)C)[C@@H]1C3 ; CCCC/C=C/C(=O)N[C@@H](Cc1cc(F)cc(F)c1)C(=O)NCCOCCOCCOCCOCCOCCOCCOCCOCCC(=O)Nc1ccc2c(C)c3c(c(O)c2c1O)C(=O)[C@]1(O)C(=O)C(C(N)=O)=C(O)[C@@H](N(C)C)[C@@H]1C3]

Further, the formulas, analogs, and R groups can be optionally substituted or functionalized with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀ alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C1-10 alkyls include, but are not limited to, -vinyl, -allyl, −1-butenyl, −2-butenyl, -isobutylenyl, -1-pentenyl, −2-pentenyl, −3-methyl-1-butenyl, −2-methyl-2-butenyl, −2,3-dimethyl-2-butenyl, 1-hexyl, 2-hexyl, 3-hexyl, -acetylenyl, -propynyl, −1-butynyl, −2-butynyl, −1-pentynyl, −2-pentynyl, or −3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O— vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O— cyclooctyl, —O-cyclononyl and —O-cyclodecyl, —O—CH2-cyclopropyl, —O—CH2-cyclobutyl, —O—CH2-cyclopentyl, —O—CH2-cyclohexyl, —O—CH2-cycloheptyl, —O—CH2-cyclooctyl, —O—CH2-cyclononyl, —O—CH2-cyclodecyl, —O—(CH2)2-cyclopropyl, —O—(CH2)2-cyclobutyl, —O—(CH2)2-cyclopentyl, —O—(CH2)2-cyclohexyl, —O—(CH2)2-cycloheptyl, —O—(CH2)2-cyclooctyl, —O—(CH2)2-cyclononyl, or —O—(CH2)2-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C3-10 cycloalkyl groups include, but are not limited to, -cyclopropyl, -cyclobutyl, -cyclopentyl, -cyclopentadienyl, -cyclohexyl, -cyclohexenyl, −1,3-cyclohexadienyl, −1,4-cyclohexadienyl, -cycloheptyl, −1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, -CH2-cyclopropyl, -CH2-cyclobutyl, -CH2-cyclopentyl, -CH2-cyclopentadienyl, -CH2-cyclohexyl, -CH2-cycloheptyl, or -CH2-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing a bacterial infection in a subject in need of administration of a therapeutically effective amount of a TDase inhibitor in combination with a therapeutically effective amount of tetracycline antibiotic compound, so as to treat a tetracycline-resistant bacterial infection.

Methods described herein are generally performed on a subject in need thereof. A subject in need of the therapeutic methods described herein can be a subject having, diagnosed with, suspected of having, or at risk for developing a bacterial infection, including tetracycline resistant bacterial infections. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a TDase inhibitor is, for example, an amount that would cause the desired therapeutic effect in a subject while minimizing undesired side effects. In various embodiments, an effective amount of a TDase inhibitor described herein can substantially inhibit bacterial TDase activation associated with tetracycline treatment of a bacterial infection, thereby slowing the progress of a bacterial infection, or limiting the development of a bacterial infection.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a TDase inhibitor can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a bacterial infection.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a TDase inhibitor can occur as a single event or over a time course of treatment. For example, a TDase inhibitor can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for bacterial infections.

A TDase inhibitor can be administered simultaneously or sequentially with another agent, such as a tetracycline antibiotic, an anti-inflammatory, or another agent. For example, a TDase inhibitor can be administered simultaneously with another agent, such as a tetracycline antibiotic or an anti-inflammatory. Simultaneous administration can occur through the administration of separate compositions, each containing one or more of a TDase inhibitor, a tetracycline antibiotic, an anti-inflammatory, or another agent. Simultaneous administration can occur through the administration of one composition containing two or more of a TDase inhibitor, a tetracycline antibiotic, an anti-inflammatory, or another agent. A TDase inhibitor can be administered sequentially with a tetracycline antibiotic, an anti-inflammatory, or another agent. For example, a TDase inhibitor can be administered before or after the administration of a tetracycline antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a TDase inhibitor, a tetracycline antibiotic, and solubilizing agents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Methods for Preparation and Use of BacPROTACS for Targeted Protein Degradation in Mycobacteria and Other Microbes

This example demonstrates a method for the preparation and use of bivalent agents for targeted protein degradation in Mycobacteria. The invention consists of two molecular entities, anhydrotetracycline (aTC) and (S,E)-(3,5-difluorophenyl)-2-(hept-2-eneamido)propanoic acid (DFP), joined via a PEG linker of variable length to form a so called BacPROTAC (aTC-PEGₙ-DFP). Also disclosed is the development of Clp-targeting chimeras (ClpTACs) that promote the degradation of TetX via the ClpP1P2 protease complex in Mycobacterium smegmatis, bypassing the need for ClpC1 chaperone involvement. The ClpTACs consist of a TDase inhibitor covalently linked to a ClpP1P2-targeting small molecule, enabling proximity-induced proteolysis of TetX and restoring tetracycline efficacy in resistant strains. Demonstrating the successful degradation of TetX-HA in M. smegmatis, providing a promising new approach for developing bacterial PROTACs targeting the ClpP1P2 complex.

### Introduction

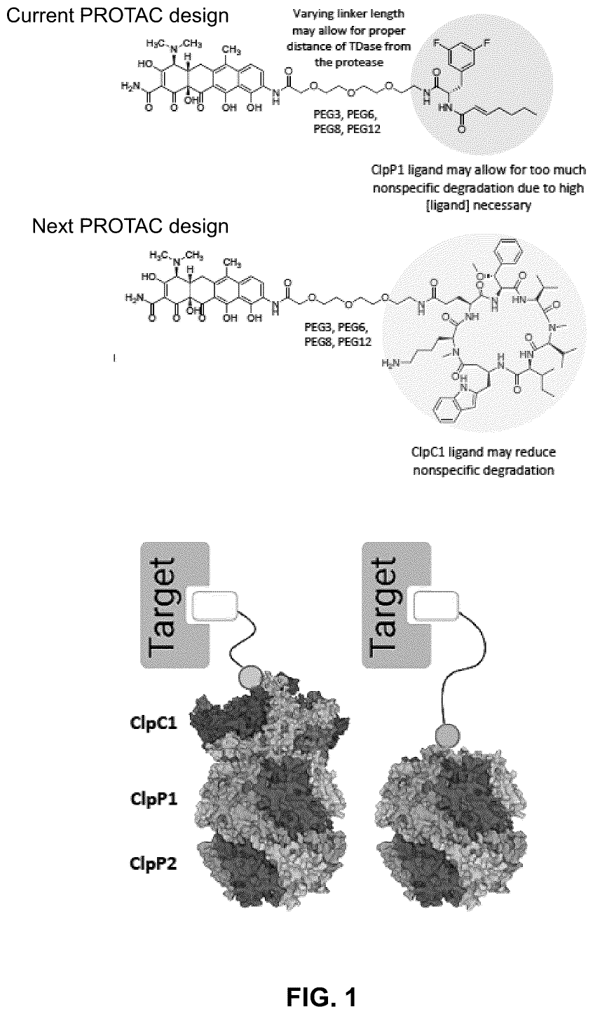

Antibiotic resistance can occur through five primary mechanisms: 1) efflux; 2) exclusion; 3) target modification; 4) metabolic reprogramming; 5) enzymatic inactivation. Antibiotic inactivating enzymes are the most problematic form of resistance in the clinic. This type of resistance requires only a catalytic amount of the enzyme and requires only a small sub-population of the cells to express the enzyme, while cells that are not resistant also benefit from this mechanism. Traditionally, this type of resistance has been managed through the development of inhibitors that are co-administered along with the antibiotic. The best example of this strategy is beta-lactam antibiotics paired with beta-lactamase inhibitors. We have previously developed inhibitors of tetracycline inactivating enzymes that serve a similar purpose. However, this method requires large concentrations of inhibitor, often resulting in toxicity and other off target effects. In this work, we reimagined how to inhibit tetracycline inactivating enzymes using targeted protein degradation (FIG. 1).

Targeted protein degradation has largely been used as a tool in anti-cancer therapies to target cancer causing proteins to the eukaryotic proteasome. These human PROTACs join a ligand targeting the protein of interest (POI) to an E3-ubiquitin ligase ligand. The net result is an increase in ubiquitylation of the POI and recruiting to the proteasome for degradation. Some bacteria including Mycobacteria, have a proteasome and utilize a similar process for protein degradation based on the prokaryotic ubiquitin like protein (Pup). The PROTAC logic from human ubiquitylation has not translated to Pupylation in bacteria due to a lack of understanding of the Pup pathway and targeting ligands for the Pup ligase. However, bacteria express some AAA+ proteases including the ClpC1P1P2 complex that can be leveraged for targeted protein degradation.

Until recently there have been few advances in the field of bacterial targeted protein degradation. There are two reported bacterial proteolysis targeting chimeras (BacPROTACs) that are effective in Mycobacteria and target the Clp chaperone-protease complex. The Clp chaperone-protease complex is essential in bacteria for intracellular protein degradation. It functions in both general protein quality control and the selective breakdown of proteins that play roles in regulating cellular processes. The most recognized substrates are proteins marked with an ssrA tag, a short peptide sequence added to the C-terminus by the tmRNA system to rescue stalled ribosomes. Clp chaperone-proteases have a cylindrical structure, composed of ATP-dependent chaperone rings that connect to a proteolytic ClpP double-ring core, enabling the degradation of substrate proteins. The interaction between the ClpP core and the chaperone is controlled by an N-terminal loop and a hydrophobic surface on the ClpP ring. Unlike Escherichia coli, Mycobacterium tuberculosis contains two ClpP protease subunits, ClpP1 and ClpP2, which form a ClpP1P2 double-ring core. This hetero double-ring structure presents two different binding surfaces for interactions with the chaperones ClpX or ClpC1. The protease active sites, located within the chamber, consist of the Ser-His-Asp catalytic triad characteristic of serine proteases. Access to this chamber is regulated by hexameric unfoldases, ClpX or ClpC, belonging to the AAA+ family (ATPases associated with various cellular activities). These unfoldases recognize protein substrates, unfold them, and then feed them into the proteolytic chamber for degradation.

Previously explored targeted protein degradation in bacteria utilized a phosphorylated guanidine, which mimics a tagged protein from a translation stalling event, or a ClpC1-targeting cyclic peptide warhead (cyclomarin) tethered to the POI ligand which dysregulates the protease and primes the POI for protein degradation. Both warheads target and activate the chaperone ClpC1 in the ClpC1P1P2 protease complex. We are using a related but unique approach to targeting TDases for degradation by the ClpP1P2 peptidase component of the protease, by employing a bivalent molecule capable of binding both ClpP1P2 and TDase to induce proximity and promote the targeted proteolysis of TDase, in a manner independent of ClpC1 chaperone binding (FIG. 1). Here, we refer to these molecules as Clp-targeting chimeras (ClpTACs). TetX is an attractive protein for these bivalent molecules because of the open channel active site that allows the accommodation of the large PEG linker and Clp-targeting ligand.

The association between the chaperone and the protease involves two conserved interaction elements. The first is an N-terminal loop located at the axial pore, through which substrates are transferred from the chaperone to the protease. The second interaction element is a hydrophobic patch on the surface of the protease ring. In chaperones ClpX and ClpC1, this patch binds to a loop containing a conserved LGF motif. Acyldepsipeptide (ADEP) antibiotics can bind to this hydrophobic patch, replacing the LGF motif and disrupting normal Clp protease function by mimicking chaperone binding.

We are currently exploring the use of a fragment of an ADEP cyclic peptide (DFP) that is known to bind and activate ClpP1P2, but this technology could also be adapted for other ClpP1P2 targeting ligands including the complete cyclic ADEPs. The ClpTAC molecule is composed of the known TDase inhibitor, aTC, joined covalently via a C9-amide bond to a ClpP1P2-targeting small molecule, (S,E)-(3,5-difluorophenyl)-2-(hept-2-eneamido)propanoic acid (DFP), through a polyethylene glycol (PEG) linker of variable length. The technology could also be adapted to target the ClpC1 chaperone component using a cyclomarin derivative in place of DFP conjugated via PEG linkage to aTC, similar to that of previously published BacPROTACs.

Here, we report the development of a ClpP1P2-targeting chimera that degrades and simultaneously inhibits TetX in M. smegmatis and rescues tetracycline activity in resistant M. smegmatis expressing TetX with a hemagglutinin (HA) tag. This represents a viable strategy for the development of future ClpP1P2-targeting bacterial PROTACs.

### Results

### Synthesis of Bivalent Ligands

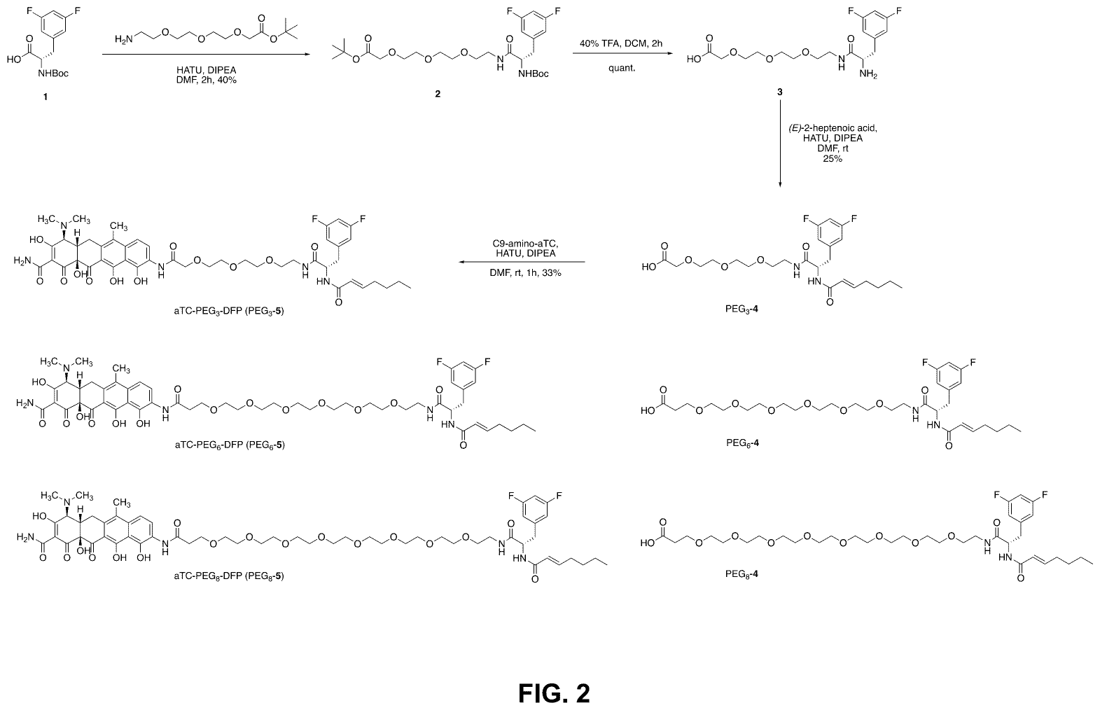

The synthesis of the bivalent ligands was completed using PEGs, PEG₆, and PEG₈ linkers (FIG. 2). N-Boc-3,5-difluoro-1-phenylalanine (1) was obtained as described previously. Treatment of compound 1 with HATU in DMF with DIPEA followed by addition of amino-PEG₃-CH₂CO₂-t-butyl-ester (BroadPharm) produced product 2 containing a new amide bond linkage. Treatment of compound 2 with anhydrous TFA in DCM gave compound 3 as the corresponding TFA salt with removal of the N-Boc and t-butyl ester protecting groups. The activation of (E)-2-heptenoic acid was achieved by treatment with HATU in DMF with DIPEA. Addition of compound 3 to this mixture resulting in the formation of amide product PEG₃-4. C9-Amino-aTC was obtained as described previously. Treatment of PEG₃-4 with HATU in DMF with DIPEA followed by addition of C9-amino-aTC provided the final bivalent compound 5, aTC-PEG₃-DFP. Two additional bivalent derivatives with longer PEG linkers, aTC-PEG₆-DFP (PEG₆-5) and aTC-PEG₈-DFP (PEG₈-5), were synthesized in an analogous manner replacing the amino-PEG₃-CH₂CO₂-t-butyl-ester reagent with amino-PEG₆-CH₂CO₂-t-butyl-ester or amino-PEG₈-CH₂CO₂-t-butyl-ester accordingly. The corresponding PEG₆-4 and PEG₈-4 intermediates were also obtained and tested in biological assays as controls. All synthetic compounds were fully characterized by NMR and purity was confirmed by LC-MS analysis prior to biological testing.

### Demonstration of Targeted Protein Degradation in Mycobacteria

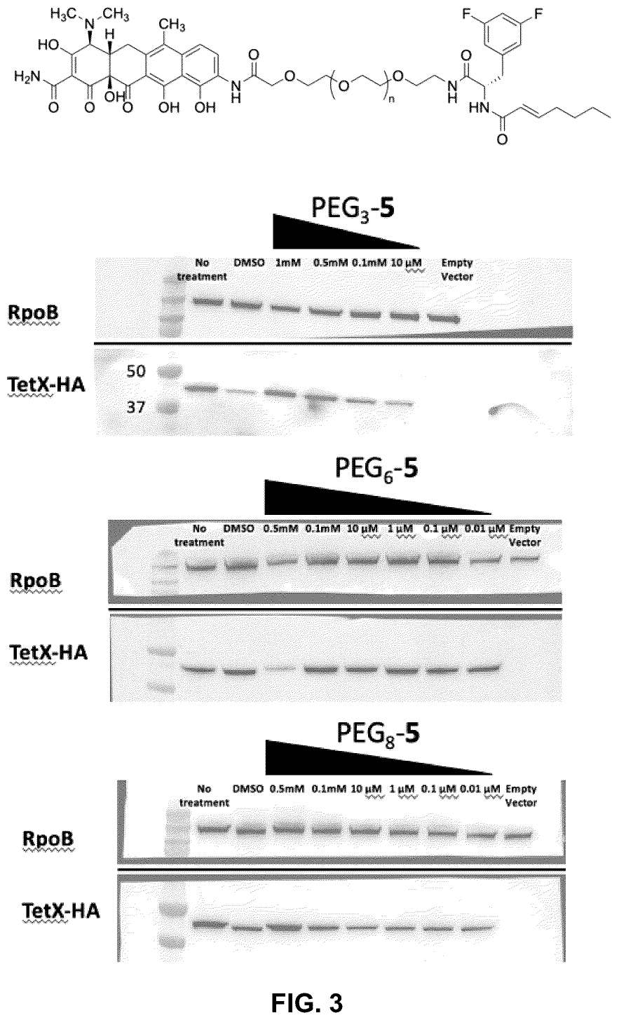

TetX-HA was expressed in Mycobacterium smegmatis for western blot visualization and quantification of dose dependent BacPROTAC-induced TetX-HA degradation (FIG. 3). Initially, PEG₃-5 was tested and used to optimize the cell treatment and immunoblotting protocols. We observed that the concentrations of DMSO used to solubilize the ligand promoted a high background of TetX-HA proteolysis. We adjusted the DMSO concentrations to decrease background TetX-HA proteolysis for the testing of PEG₆-5 and PEG₈-5 (FIG. 3). The immunoblots revealed that the ClpTACs can have a protective effect or degradative effect on TetX-HA depending on the concentration and length of the PEG linker. The observed protective effect (see PEG₃-5 1 mM concentration and PEG₈-5 0.5 mM concentrations) could be due to stabilization of TetX-HA through binding to the aTC component of the bifunctional ligand, unfavorable proximity in the ClpP1P2/TetX-HA/ClpTAC ternary complex, or competitive blocking of ClpC1 due to the DFP ligand binding to ClpP1P2.

The PEG₆-5 compound showed the most dramatic induced protein degradation at 0.5 mM ClpTAC which inspired us to further investigate the mechanism for inducing TetX-HA degradation by this compound using in vitro assays with purified ClpC1P1P2 from Mycobacteria. Clearly, the nature of the ClpTAC linker is important for achieving optimal TDase degradation in Mycobacteria, and various linkers should be compared in future studies. For the remainder of this study, we focused on the detailed characterization of PEG₆-5 which showed the most promising results in M. smegmatis.

### Demonstration of Targeted Protein Degradation In Vitro

The genes encoding for ClpP1P2C1 were cloned from Mycobacterium tuberculosis into a pET21 plasmid to enable heterologous expression in E. coli and purification by affinity and size exclusion chromatography.

### BacPROTACs Recover Tetracycline Activity in Mycobacteria Expressing TDase

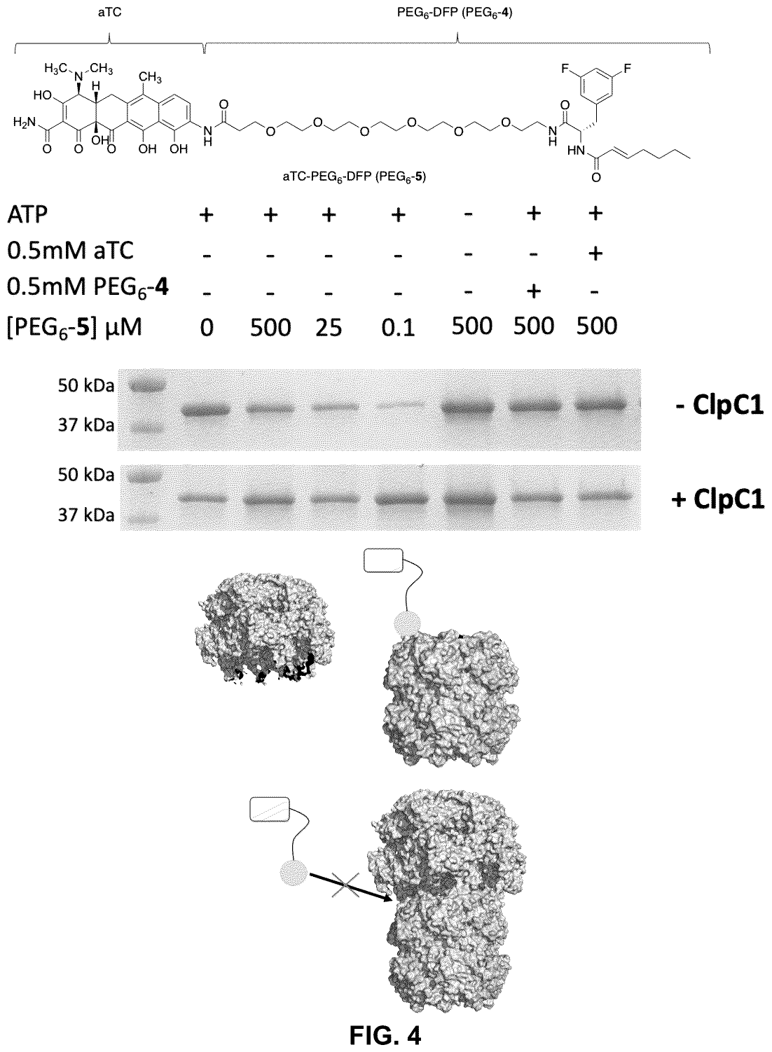

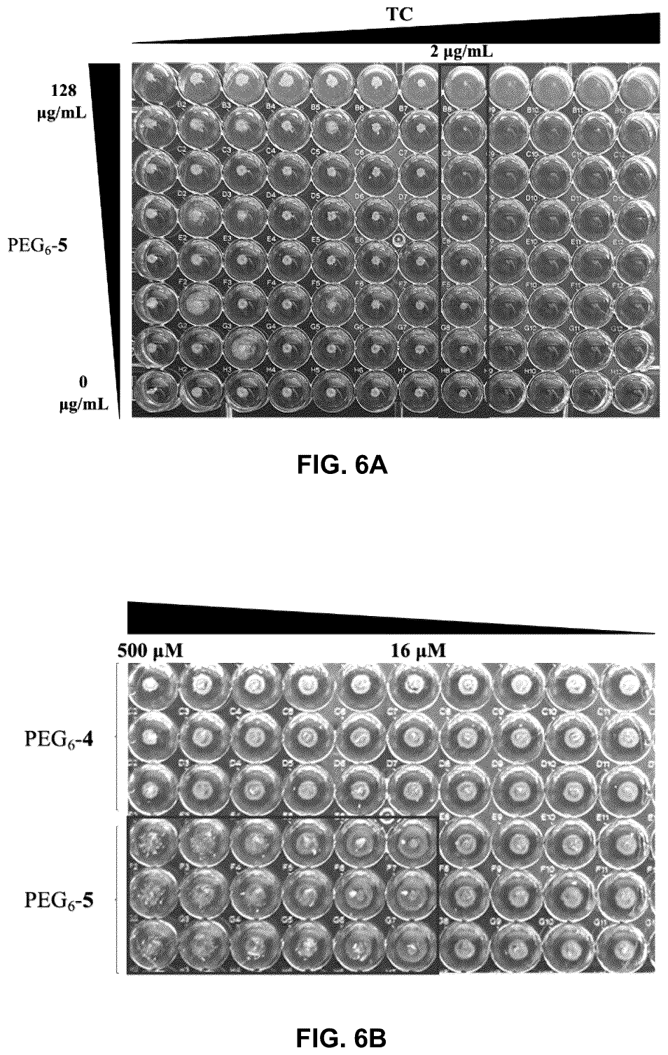

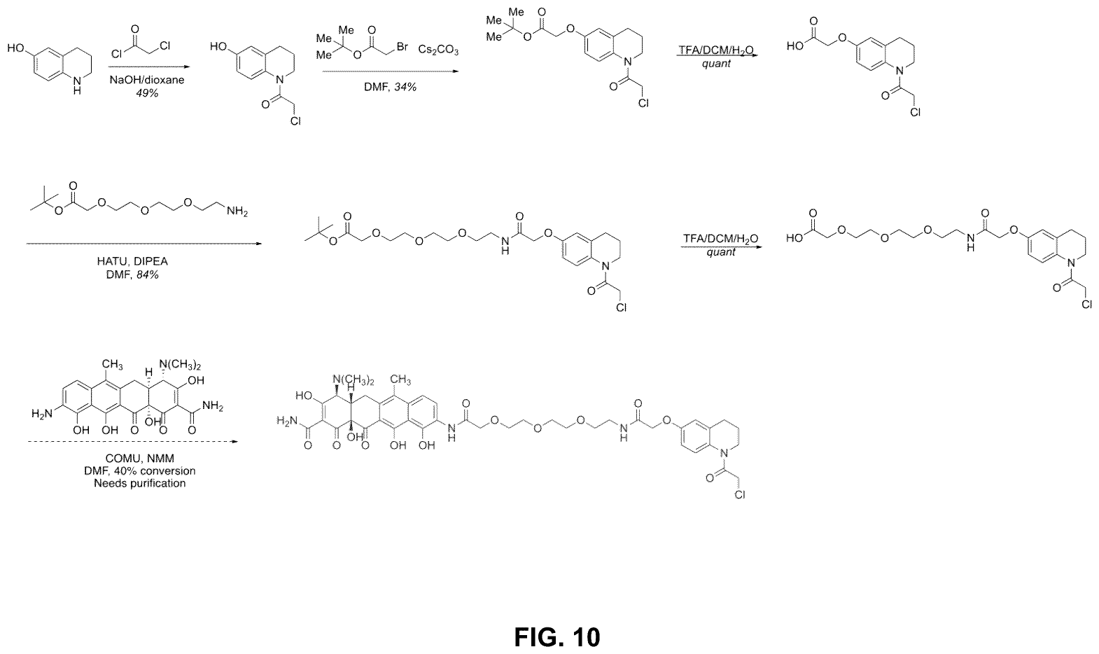

The ability of BacPROTAC PEG₃-5 to rescue the antibacterial activity of tetracycline was tested against M. smegmatis expressing TetX. The M. smegmatis strain expressing TetX showed a clear tetracycline resistance phenotype with MIC₉₀ values of 0.5 μM for empty vector control and 16 μM for TetX expression from the plasmid vector (Pmsg430 or Pmv261). In the presence of fixed tetracycline at 8 μM, the rescue of tetracycline activity was seen at 256 μM BacPROTAC PEG₃-5 (FIG. 6). The BacPROTAC PEG₃-5 alone displayed no growth inhibitory activity towards the M. smegmatis strains. To form the mature and functional ClpP particle, ClpP1 and ClpP2 assemble into a ClpP1P2 double-ring, composed of one homoheptameric ring from each subunit. In vitro, this assembly requires an activator peptide to generate a functional protease. The activator peptide, such as Z-Leu-Leu-H (Benzyloxycarbonyl-L-Leucyl-L-Leucinal), or a similar molecule, binds near the active sites of the proteolytic particle, stabilizing the active conformation of the ClpP1P2 double-ring. The presence of ClpC1 and a protein substrate further stabilizes this functional conformation, working synergistically with the activator peptide. We optimized combinations of ClpP1P2 with or without ClpC1 for activity using Z-Leu-Leu-H. We confirmed the peptidase activity of purified ClpC1P1P2 using a peptide tagged with 7-Amino-4-methylcoumarin (AMC) as a fluorescent probe (FIG. 11). Using these optimized reaction conditions, we monitored for proteolysis of purified TetX in the presence of varied PEG₆-5 BacPROTAC, ClpP1P2, ClpC1P1P2, cosubstrates (ATP). We observed near complete proteolysis of TetX after 6 hours under full reaction conditions using ClpP1P2 and ClpC1P1P2, while reactions lacking ATP showed less proteolysis of TetX (FIG. 4). The 3-hour timepoint revealed a clear promotion of TetX proteolysis by PEG₆-5 ClpTAC at 0.1 μM in the presence of ClpP1P2 with a reduction in apparent proteolysis at higher concentrations in comparison to background proteolysis by ClpP1P2. The inclusion of the chaperone ClpC1 reduced the amount of observed TetX proteolysis in the presence of the ClpTAC, consistent with the concept that ClpC1 can compete with the ClpTAC PEG₆-5 for binding to the ClpP1P2 protease via the DFP moiety. As expected, aTC and PEG₆-4 appear to antagonize the ClpTAC activity, and treatment with aTC and PEG₆-4 separately without ClpTAC do not induce degradation of TetX.

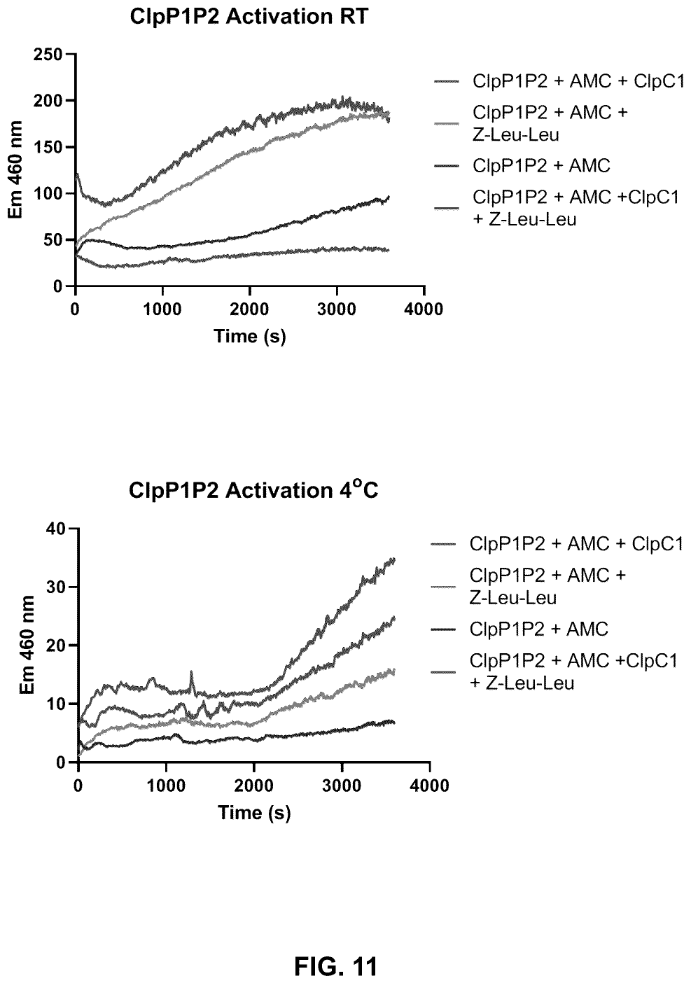

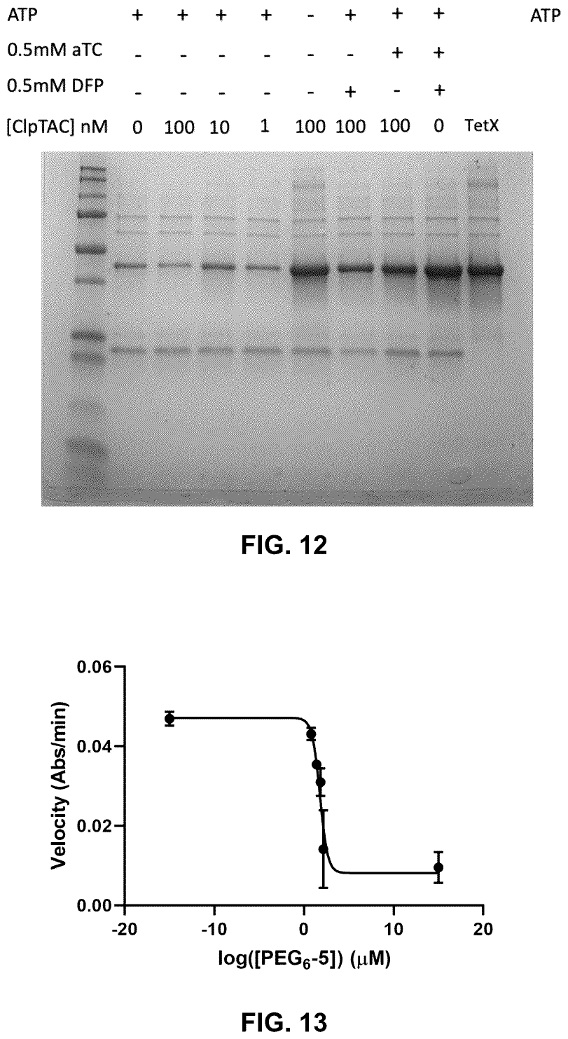

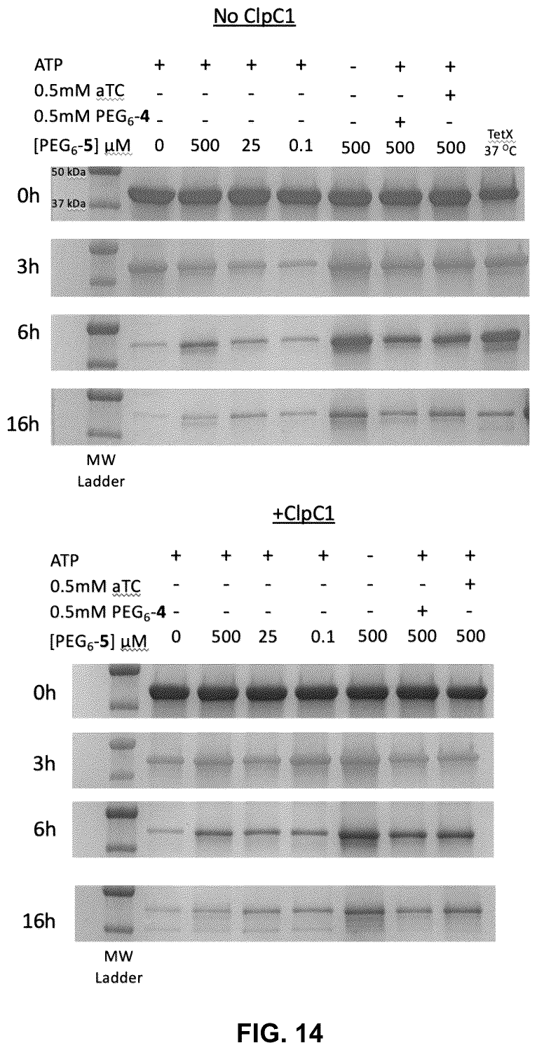

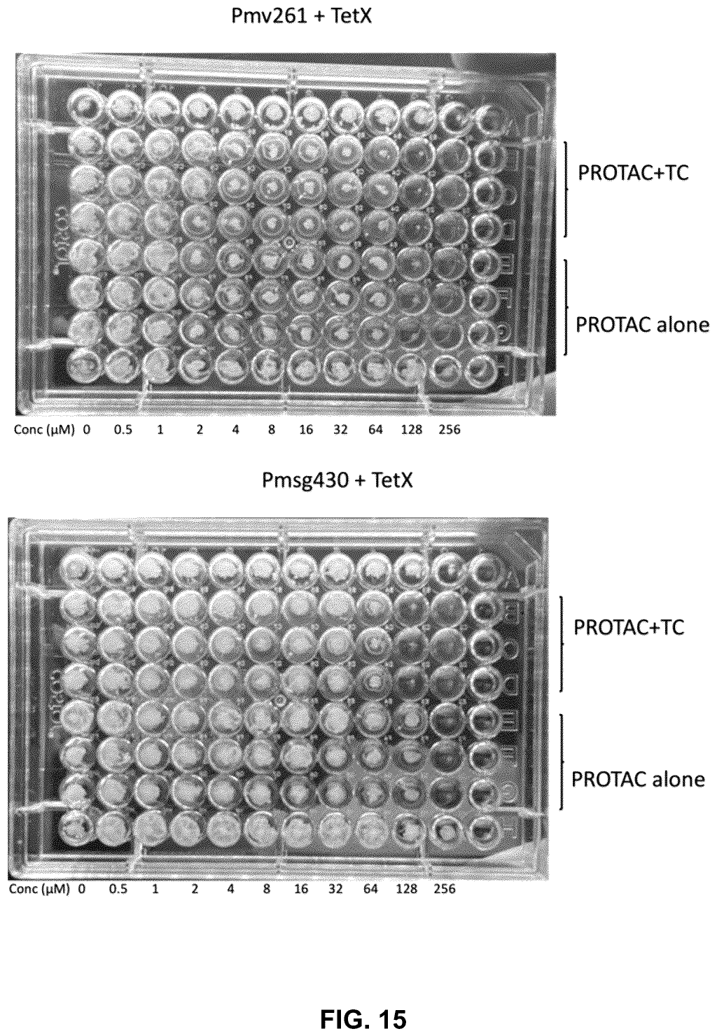

Higher concentrations of PEG₆-5 have a protective effect as observed in the Mycobacteria cell-based assays. Hence, we conclude that lower concentrations of PEG₆-5 (100 μM or less) are optimal for achieving targeted degradation of TetX by ClpP1P2 using our ClpTAC approach in vitro, but higher concentrations are needed in cells to achieve optimal diffusion into cells. Higher concentrations may also be needed to out compete ClpC1 in the cell, whereas lower concentrations are needed in our in vitro experiments due to the artificial exclusion of ClpC1. This may represent a benefit to targeting ClpC1 rather than the ClpP1P2 peptidase component, but future studies are required for direct comparison. Controls containing 0.1 μM ClpTAC are in FIG. 12. We also confirmed that TetX is inhibited by PEG₆-5 with an IC₅₀ of 61.2±19.5 μM (FIG. 13).

### ClpTACs Recovery of Tetracycline Activity in Mycobacteria Expressing TDase

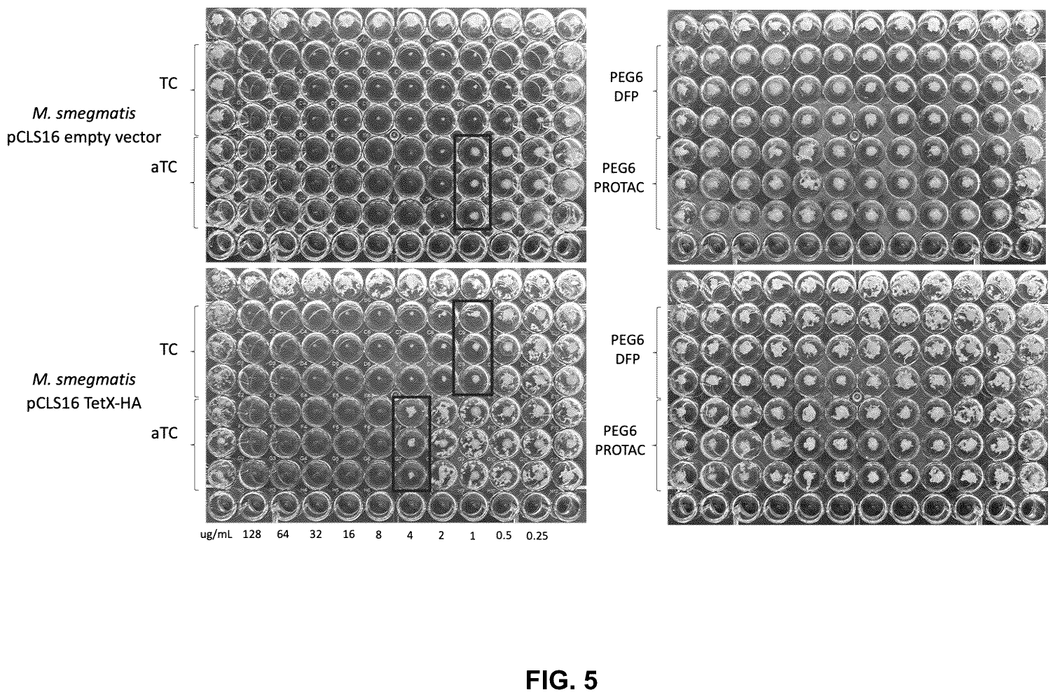

To test the ability of ClpTAC PEG₆-5 to rescue TC in TetX-expressing Mycobacteria, we first confirmed that the M. smegmatis cells expressing TetX-HA are more resistant to TC compared to the pSCL16 empty vector (EV) cells and found that the Minimum Inhibitory Concentration (MIC)¹⁹ increased from 0 μg/mL TC (EV) to 1-2 μg/mL TC (TetX-HA) and from 1-2 μg/mL aTC (EV) to 4-8 μg/mL aTC (TetX-HA). We also confirmed that PEG₆-4 and PEG₆-5 do not have any intrinsic antibacterial activity up to 128 μg/mL (FIG. 5). To monitor the effect of TetX degradation on TC rescue, we performed Fractional Inhibitory Concentration Index (FICI) analysis of PEG₆-5 and TC (FIG. 6). We observed PEG₆-5 concentration dependent rescue of TC at 2 μg/mL TC. Therefore, we chose 2 μg/mL TC for measuring MICs of PEG₆-4 and PEG₆-5, and observed rescue of TC activity between 250 μM to 500 μM of PEG₆-5, and no dose dependent response with PEG₆-4, indicating that the full aTC-PEG-DFP conjugate is needed.

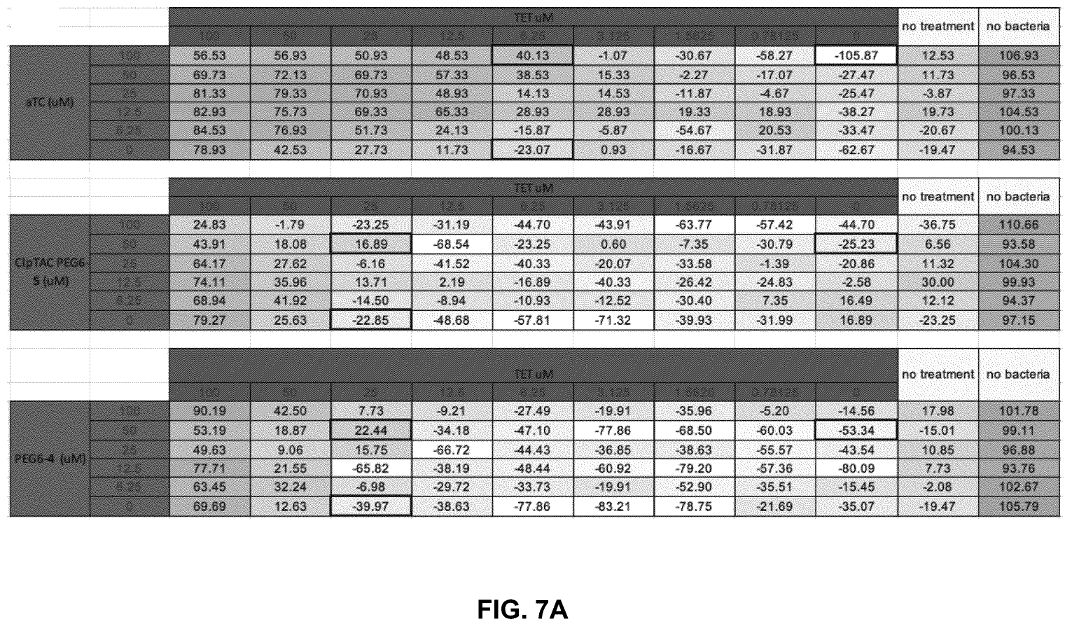

The activity of ClpTAC PEG₆-5 compared to control molecules aTC and PEG₆-4 were evaluated using a ‘checkerboard’ synergy assay in combination with tetracycline (TE) against Mycobacterium abscessus L948. M. abscessus naturally produces a TetX homologue that causes resistance to tetracycline antibiotics.²⁰ This strain presents a real-world challenge for overcoming TetX-mediated resistance using the ClpTAC approach on a clinical isolate. Several concentrations of aTC/Tet, ClpTAC PEG₆-5/Tet, and PEG₆-4/Tet produced a growth inhibitory effect that was more than additive suggesting the ClpP1P2 activation is synergistic with overcoming TetX-mediated resistance to Tet antibiotics (FIG. 7). The observed synergy of the control compound aTC, a known TetX inhibitor, was as expected based on our prior reports of aTC/Tet combinations overcoming TetX-mediated resistance.²¹ The activities of ClpTAC PEG₆-5 and control compound PEG₆-4 revealed distinct patterns of dose-dependence. The control compound PEG₆-4 showed optimal synergy at the highest tested concentration of 100 μM while ClpTAC PEG₆-5 had optimal activity at the lowest concentrations tested of 6.25 and 12.5 μM. These findings are consistent with a model were general ClpP1P2 activation by the acyldepsipeptide fragment increases TetX degradation while proximity induction by the bifunctional ClpTAC PEG₆-5 molecule requires a lower concentration to avoid competition with ClpX and the prozone effect which can isolate the two targets and limit the desired proximity-induced proteolysis.

### Discussion and Conclusions

We report a new approach to overcoming antibiotic resistance via targeted proteolytic degradation of antibiotic inactivating enzymes. Specifically, we explored the targeted degradation of TetX in vitro and in whole cell M. smegmatis expressing TetX-HA. This approach uses the previously developed TDase inhibitors, C9-substituted aTC analogs, as a targeting agent which is covalently attached to a protease targeting ligand, a fragment of an acyldepsipeptide (DFP), through a variable length PEG linker. The chimeric aTC-PEG-DFP simultaneously inhibits TDase and recruits it to the ClpP1P2 protease to promote targeted proteolysis of the TDase through induced proximity and activation of the protease. This approach is unique, as it uses a bifunctional molecule that binds and simultaneously inhibits TetX, while inducing proximity to and activating the ClpP1P2 protease. Our protease targeting ligand competes with ClpC1 chaperone binding to the ClpP1P2 protease, allowing the protease to degrade surrounding proteins without the need of its unfoldase chaperone component. We were able to visualize degradation in cells using western blot analysis, and in vitro using pure proteins and SDS PAGE gel analysis. This approach is a promising route to overcoming resistance by TDases, as the combination of a TC antibiotic and our PROTAC molecule, is simultaneously able to degrade and inhibit TDase. Future studies will compare the ADEP fragment (DFP) with a full cyclic ADEP to see if more potent ClpP1P2 binding and activation are required improve the proximity-induced proteolysis of TetX. Further, we will explore if the aTC component stabilizes TetX and slows its proteolysis by ClpP1P2 which has been shown for dihydrofolate reductase inhibitors.

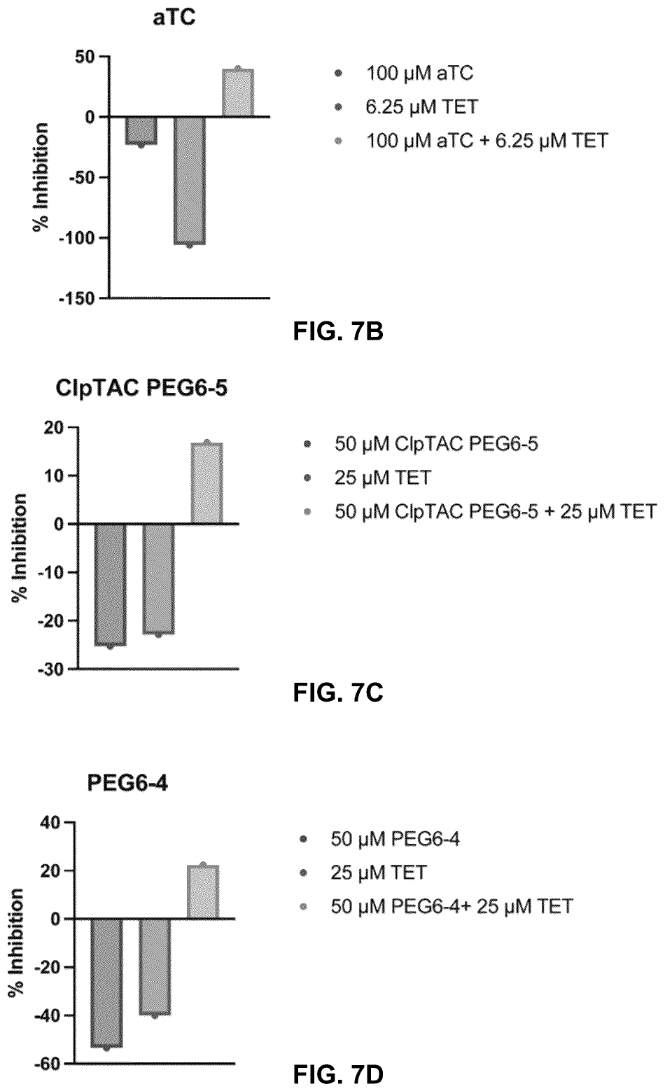

Bacterial PROTACs are in the early days of development, and there are many future directions for targeted protein degradation in bacteria. There is still much room for improvement in targeting the Clp protease system, including new ligands and expanding the proteins that are targeted for degradation. The ClpC1-targeting ligand developed by the Clausen group¹⁰ can be linked to aTC to compare to the ClpP1P2 targeting approach. The synthesis involves solid phase peptide synthesis (SPPS), and requires that two noncommercially available amino acids, Fmoc-L-Phe(3R-MeO)—OH and aTC-PEG, are synthesized, one of which has already been synthesized by Dr. Ruihao Li in the Wencewicz group (FIG. 8). Synthesis of Fmoc-L-Phe(3R-MeO)—OH was achieved in several steps. First, L-threo-Phenylserine was treated with phthalic acid anhydride to protect the amine, then the hydroxyls were methyl protected using proton sponge and Me₃OBF₄. The phthaloyl was removed by treatment with hydrazine, and the N-Fmoc was formed by treatment with Fmoc-OSu and base. To prevent concomitant cleavage of the Fmoc protecting group during ester hydrolysis, the Nicolaou protocol for methyl ester deprotection was used. Next, we will use SPPS to build the cyclomarin derivative. In addition to the cyclomarin derivative, which targets ClpC1, synthesizing the full cyclic acyldepsipeptide that targets ClpP1P2 may also allow us to use less PROTAC compound and enhance TDase degradation. These approaches can also be expanded to other AMR-causing enzymes, such as beta-lactamases, by replacing the aTC component with a beta-lactamase inhibitor, such as clavulanic acid.

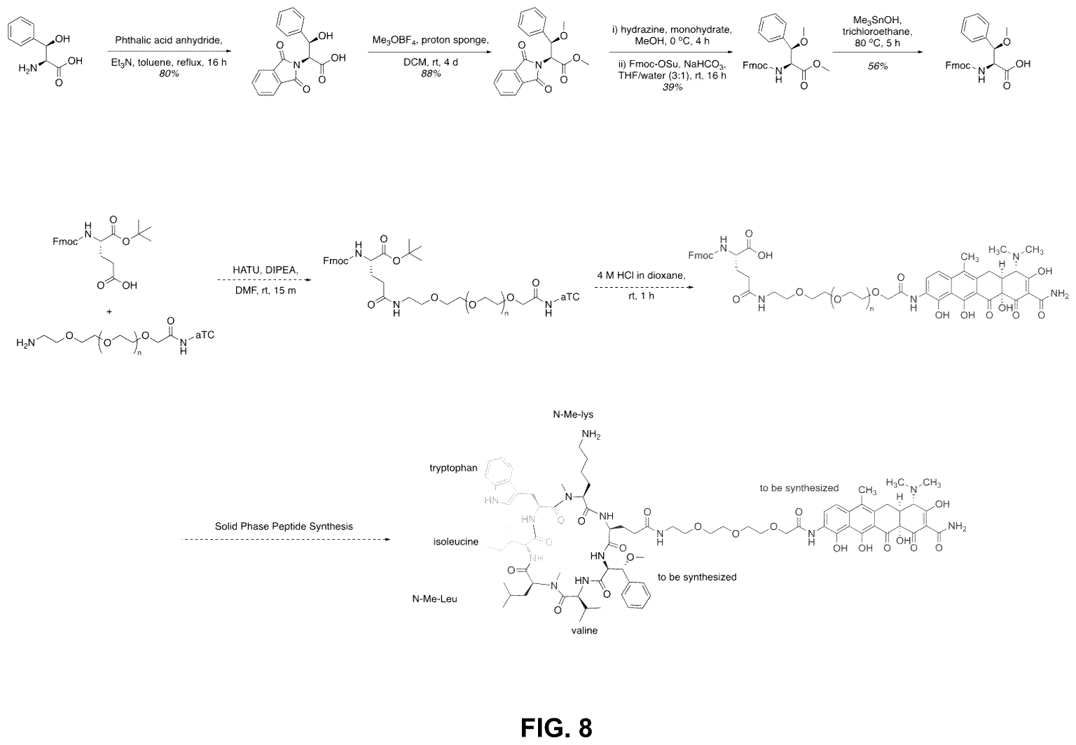

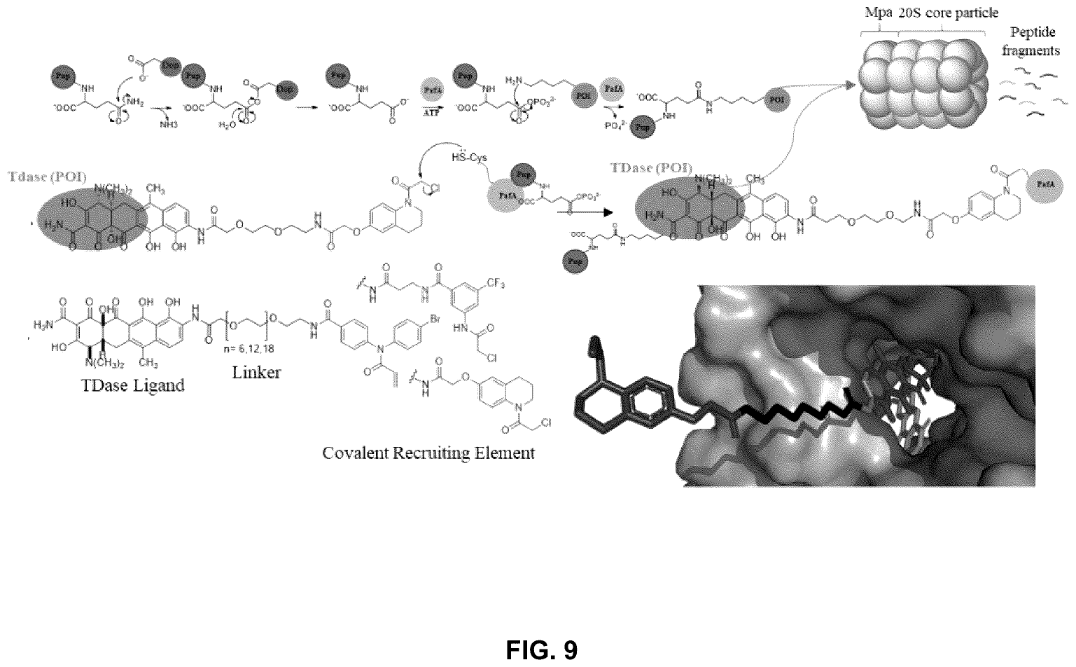

Other pathways in the bacterial protein degradation machinery can theoretically be targeted, such the pup-proteasome system (FIG. 9). However, these pathways do not currently have ligands that can be used in a PROTAC molecule. Proteomics or screening efforts would be needed to find ligands for these pathways. One potential approach is to use cysteine reactive electrophilic “scout” molecules to bind to the pup ligase and induce pupylation of the protein of interest. The ‘scouting’ approach was originally developed by Dr. Ben Cravatt and coworkers, and this method was used to assay targets of interest directly in human cell proteomes without requiring protein purification. Previous efforts in the lab have resulted in the synthesis of aTC joined through a PEG linker to an electrophilic scout fragment, but future work is needed to develop the biological assays to test this method. The synthesis involves synthesizing the cysteine-reactive probe then coupling it to aTC-NH2 via COMU-mediated coupling or HATU-mediated coupling as previously discussed (FIG. 10). This method could be expanded by linking to other electrophilic scout molecules.

### Experimental Methods

All organic solvents including deuterated NMR solvents and reagent chemicals used in preparation or analysis of synthetic compounds were obtained commercially and used without further purification. aTC (HCl salt) was purchased from Chemodex (United Kingdom). NMR spectra were obtained on a Varian Unity-Inova 500 MHz or Agilent Premium Compact+600 MHz spectrometer in 5 mm type 1, class A borosilicate glass NMR tubes (Wilman LabGlass part No. 535-PP-8). All free induction decay files (FIDs) were processed using Mestrenova version 11.0.4 software. Chemical shifts (6) are reported in parts per million (ppm) and referenced to residual non-deuterated solvent. Coupling constants (J are reported in hertz (Hz). LC-MS analysis was performed using an Agilent 6130 single quadrupole instrument (ESI+) with G1313 autosampler, G1315 diode array detector, and 1200 series solvent module with separation on a Phenomenex Gemini C18 column, 50×2 mm (5 μm) fit with a guard column cassette. LC-MS solvents were 0.1% formic acid in H₂O (A) and 0.1% formic acid in ACN (B). Solvent gradient was linear starting from 0% B to 95% B over 20 min at a flow rate of 0.5 mL/min. HPLC was performed on an HP1050 system using a Luna 10 mm C18(2) 100 Å column (250 mm×21.2 mm) from Phenomenex fit with a guard column of the same matrix (15 mm×21.2 mm). HPLC solvents were 0.1% formic acid in H₂O (A) and 0.1% formic acid in ACN (B) with a gradient formed from 0% B to 95% B over 20 min at a flow rate of 9 mL/min. LC-MS and HPLC data were processed using ChemStation software version B.04.02 SP1. Liquid medium bacterial growth assays were performed using Mueller-Hinton or LB broth in Costar 96-well plates at 37° C. End-point growth density was judged by OD600 measurement using a Tekan Infinite 200 Pro M Plex plate reader. Fluorescence measurements were performed in flat-bottom, black, opaque, polystyrene 96-well Microfluor 2 microplates (Thermo Fisher, Inc). SDS-PAGE analysis was carried out using Bio-Rad Any kD precast polyacrylamide gels with staining by Coomassie brilliant blue and comparison to a Bio-Rad precision plus protein dual Xtra pre-stained protein standard ladder.

### Protein Expression and Purification

Plasmid DNA (pET21) was transformed into E. coli BL21 (ClpP1-His and ClpP2-His) or Rosetta cells (ClpC1-His) and grown in LB broth supplemented with 50 μg/mL ampicillin at 37° C. Protein expression was induced by adding 1 mM IPTG at an OD₆₀₀ of 0.8 and shaking at 225 rpm overnight at 15° C. The cells were harvested by centrifugation and lysed by sonication in a buffer containing 500 mM NaCl, 50 mM TRIS pH 7.5, 10 mM imidazole, and 0.25 mM TCEP. The lysated cells were incubated with Ni-NTA resin. After 2 40-mL washes with the suspension buffer, protein was eluted with 50 mM TRIS, 100 mM NaCl, 300 mM imidazole, and 0.25 mM TCEP. Protein was dialyzed into 50 mM TRIS, 300 mM NaCl overnight at 4° C., concentrated to 2 mL. ClpP1 and ClpP2 were flash frozen and stored at −80° C. ClpC1 was loaded onto the FPLC using a Cytiva Superdex 200 Increase 10/300 GL column and eluted with 50 mM TRIS and 300 mM NaCl buffer. ClpC1-containing fractions were pooled, concentrated, and flash frozen. TetX was purified with an N-terminal His6 tag.

### In Vitro TetX Degradation Assays

ClpP1P2 or ClpC1P1P2 was preactivated by mixing equal volumes of 2.5 mg/mL ClpP1 and 2.5 mg/mL ClpP2, and 30 μM Z-Leu-Leu-H at 25° C. Equal volume of 2.5 mg/mL ClpC1 was added for experiments containing ClpC1. In vitro degradation assays containing 15 μg/mL ClpP1, 15 μg/mL ClpP2, 2 mM ATP, and 0.5 mg/mL TetX were performed in a buffer containing 100 mM PBS pH 7.5 (at 37° C.) and 5% (v/v) glycerol. 15 μg/mL ClpC1 was added to reactions containing ClpC1. Compounds were dissolved in 100% (v/v) DMSO and further diluted to a final concentration of 2.5% (v/v) DMSO in the assay. 2.5% (v/v) DMSO was also added to control experiments. Reactions were quenched by removing 20 μL sample and adding to 5 μL SDS sample buffer. Samples were analyzed by SDS-PAGE and Coomassie blue staining.

Western Blot Visualization of TetX-HA Degradation in Mycobacterium smegmatis

100 mL cultures of Mycobacterium smegmatis (pCLS16 vector) with 20 μg/mL kanamycin were grown in LB broth supplemented with 0.5% dextrose, 0.5% glycerol, and 0.05% Tween80. The cells were back diluted to an OD₆₀₀ of 0.4 in 7.6 mL broth and treated with the 400 μL of the corresponding concentration of BacPROTAC (PEGₙ-5, dissolved in DMSO at varying concentrations to give same final v/v DMSO. After 4 hours of treatments, cells were centrifuged to give a final OD₆₀₀ of 14 in 250 μL of lysis buffer containing 50 mM TRIS HCl (pH=8.0), 150 mM NaCl, and 1×EDTA free protease inhibitor. Cells were lysed by bead beating then centrifuged for one minute, followed by supernatant removal and 250 μL of SDS sample buffer with 2 mM DTT added. Samples were boiled in a heat block at 90° C., then pelleted at 5000 rpm for 30 seconds before loading on to NuPAGE Invitrogen 4-12% gel using MOPS buffer. Gel was equilibrated in 2× Biorad transfer buffer for 10 min then 50 mL of 40% methanol in water was added to the soaking gel before nitrocellulose transfer at 15 V for 1 hour. Membrane was incubated with 5% skim milk in 1×TBST for 1 hour then treated with mouse anti-RpoB or mouse anti-HA at 4° C. overnight. Blots were washed with TBST buffer three times then incubated with goat anti-mouse antibody for 1 hour at room temperature. Blots were washed three time with TBST then developed using Cytiva ECL Prime Western Blotting Detection Kit.

Mycobacterium smegmatis Growth Inhibition

MIC₉₀ values were determined using standard methods by the broth microdilution method. MIC panels were prepared in 96-well flat-bottom microplates (Corning) by two-fold serial dilution of inhibitors in LB broth supplemented with 0.5% dextrose, 0.5% glycerol, and 0.05% Tween80 supplemented with 20 μg/mL kanamycin (KAN20). M. smegmatis containing TetX-HA or empty vector pCLS16 were grown for approximately 48 hours to an OD₆₀₀ of 0.2-0.8, then diluted to an OD600 of 0.05. The diluted cells were inoculated into the MIC panel at a 1:1 ratio. The panels were incubated at 37° C. for 48 h, then scored by visual inspection. The FICI studies were completed analogously using variable concentrations of test compounds in combination with tetracycline as shown in FIG. 6.

Mycobacterium abscessus Growth Inhibition

Inhibition of Mycobacterium abscessus growth was evaluated using the MABA assay as following a general protocol. Briefly, A 10 mL culture of M. abscessus L948 was grown at 37° C. while shaking at 150 rpm to logarithmic phase (OD₆₀ between 0.2-0.8) in 7H9 media supplemented with 10% ADC and 0.05% glycerol. The culture was diluted to OD₆₀₀=0.01 in 7H9 media and 100 μL of it was inoculated into clear, flat-bottomed 96-well plates (Fisher Scientific, CAT #08-772-53) containing an equal volume of test compounds at 2× their final concentration in 7H9 media. DMSO from the compound stocks was kept at a final concentration of 1% in all wells. After incubation at 37° C. for 48 hr, 32.5 μL of a resazurin/Tween 80 mixture (8 parts 0.6 mM resazurin in 1×PBS and 5 parts 20% Tween 80 in water) was added to each well and the plates were allowed to incubate for an additional 16 hr. After incubation was completed, pictures were taken, fluorescence intensity at λₑₘ=590 nm (λₑₓ=530 nm) was measured using a BioTek Synergy H1 microplate reader, and relative fluorescence intensity used to calculate % inhibition. A plot of % inhibition, with the no bacteria control serving as 0% inhibition and the no test compound control serving as 100% inhibition, was used to calculate IC₅₀ by nonlinear regression analysis in GraphPad Prism 10. Testing for all compounds was performed in triplicate as independent trials.

### Synthesis

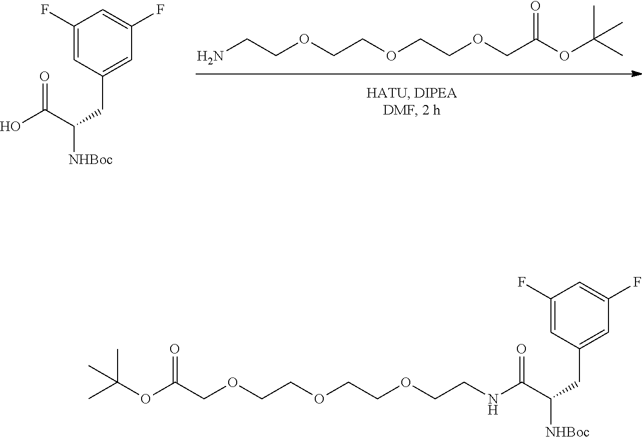

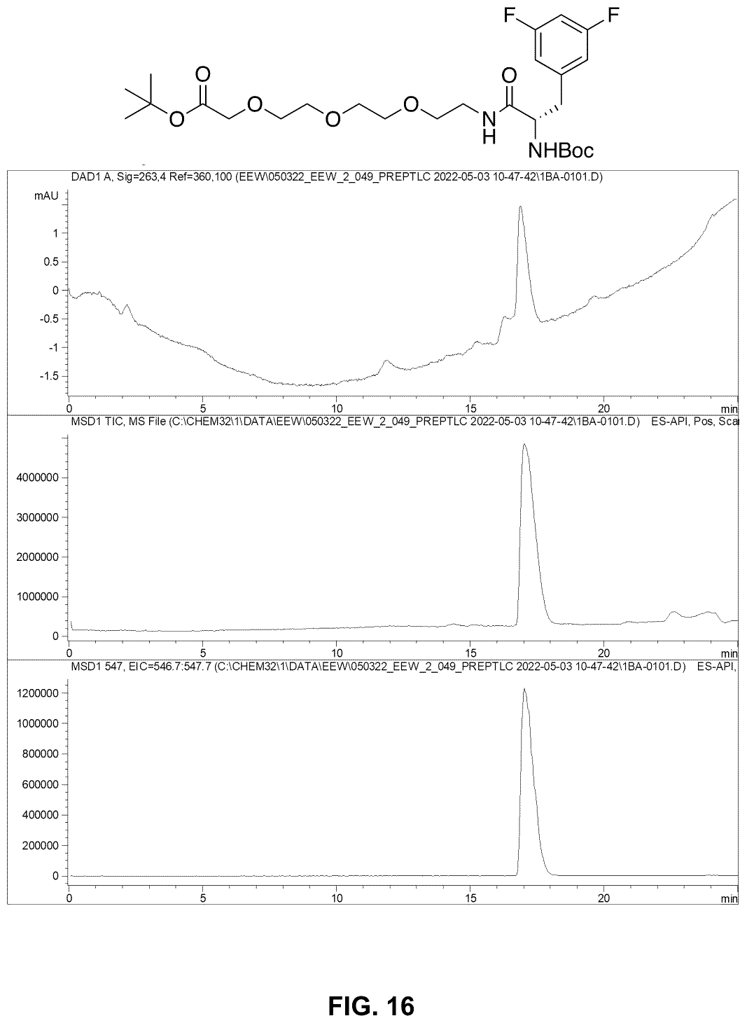

N-Boc-difluorophenylalanine (1, 292.3 mg, 0.97 mmol) was dissolved in 3.5 mL anhydrous DMF and stirred for 5 min at room temperature with DIPEA (33.79 μL, 0.19 mmol) and HATU (503.0 mg, 1.32 mmol). Reaction transferred to vial containing amino-PEG3-CH₂CO₂-t-butyl-ester (244.0 mg, 0.93 mmol) and the colorless solution turned yellow. Reaction stirred for 2 hours at room temperature then diluted with ethyl acetate and washed with 1 M HCl (3×15 mL), followed by saturated sodium bicarbonate (2×15 mL). The organic layer was dried over sodium sulfate and the solvent was removed by rotary evaporation. The resulting yellow oil was purified by preparative TLC using 80:20 ethyl acetate:hexanes (R_{f}=0.6). The product was extracted from the silica using ethyl acetate, and the solvent was removed by rotary evaporation, yielding 223 mg (0.41 mmol, 44%) of desired product 2 (FIG. 16). MS (ESI+): [M+H]⁺; found, 547.0.

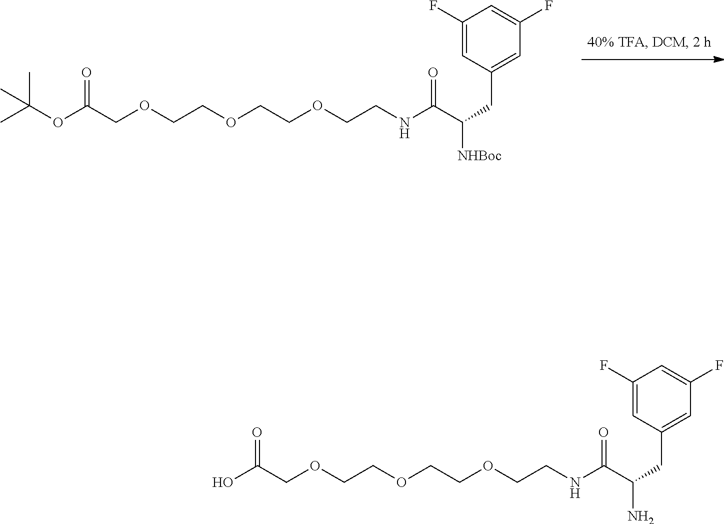 [SMILES: CC(C)(C)OC(=O)COCCOCCOCCNC(=O)[C@H](Cc1cc(F)cc(F)c1)NC(=O)OC(C)(C)C ; N[C@@H](Cc1cc(F)cc(F)c1)C(=O)NCCOCCOCCOCC(=O)O]

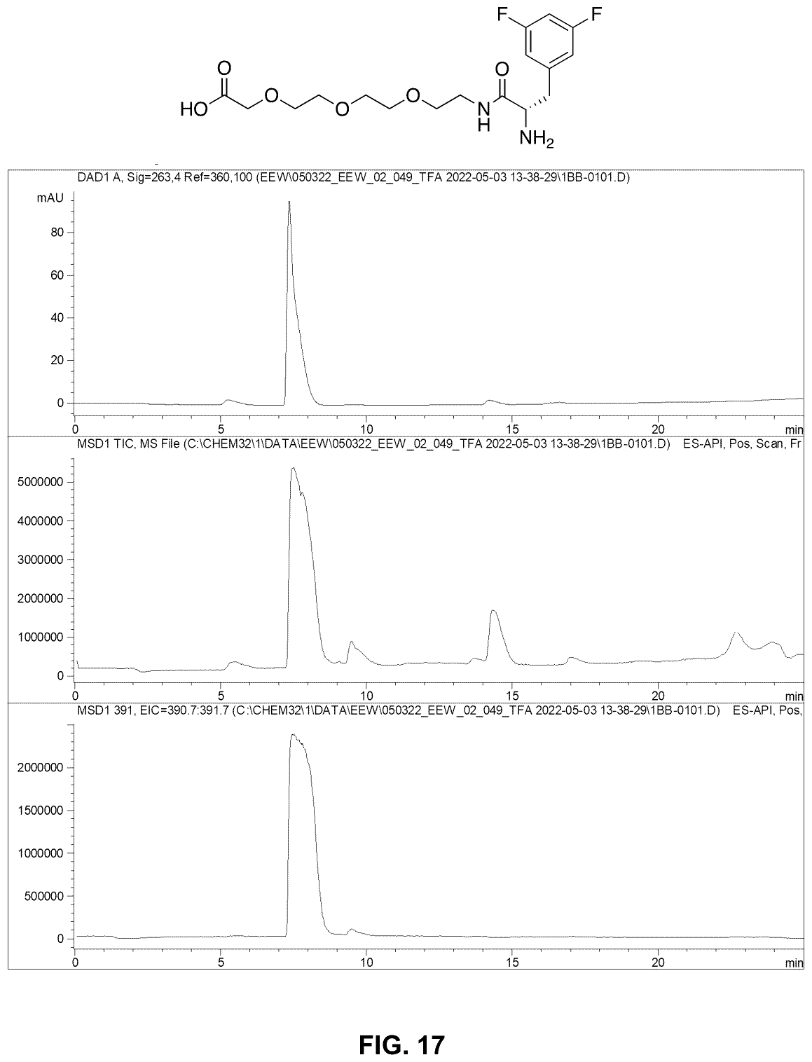

Compound 2 (223 mg, 0.41 mmol) was dissolved in 0.6 mL DCM and 0.4 mL TFA and stirred at room temperature for 1.5 hours, yielding 159 mg (0.41 mmol, quant.) of desired product 3 as the corresponding TFA salt. MS (ESI+): [M+H]⁺; found, 391.1 (FIG. 17).

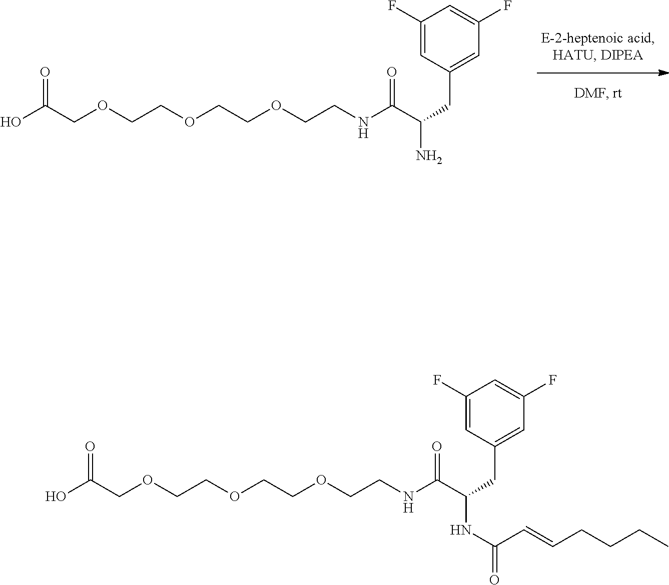 [SMILES: N[C@@H](Cc1cc(F)cc(F)c1)C(=O)NCCOCCOCCOCC(=O)O ; CCCC/C=C/C(=O)N[C@@H](Cc1cc(F)cc(F)c1)C(=O)NCCOCCOCCOCC(=O)O]

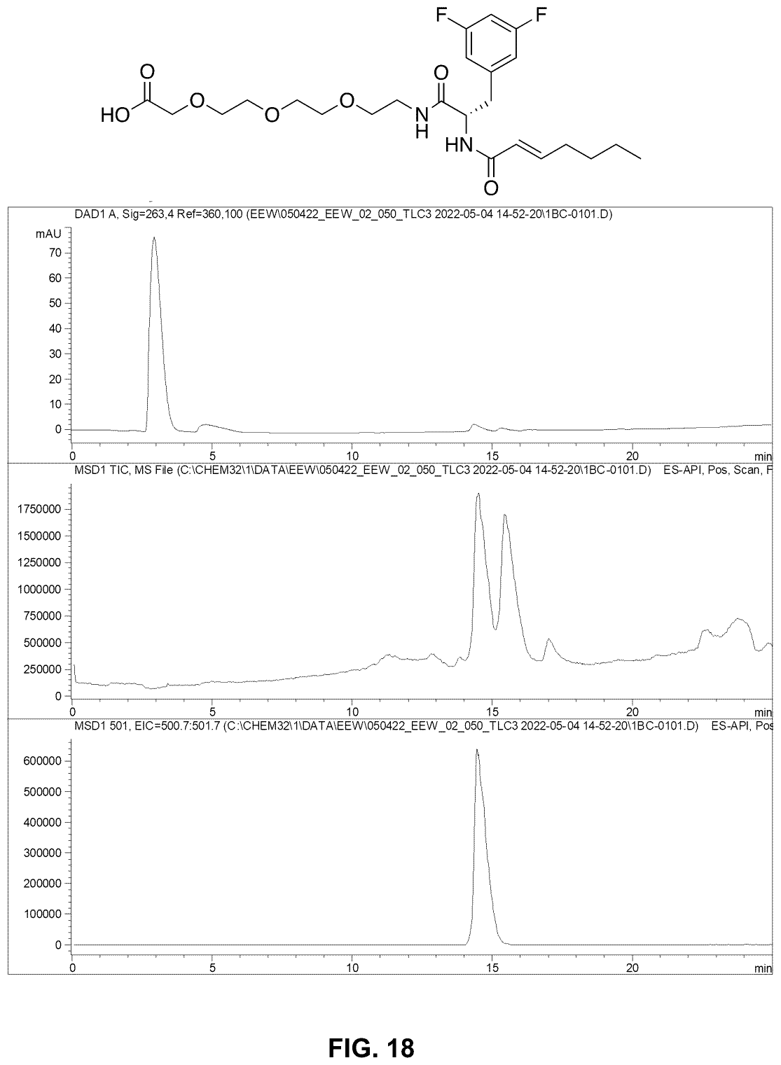

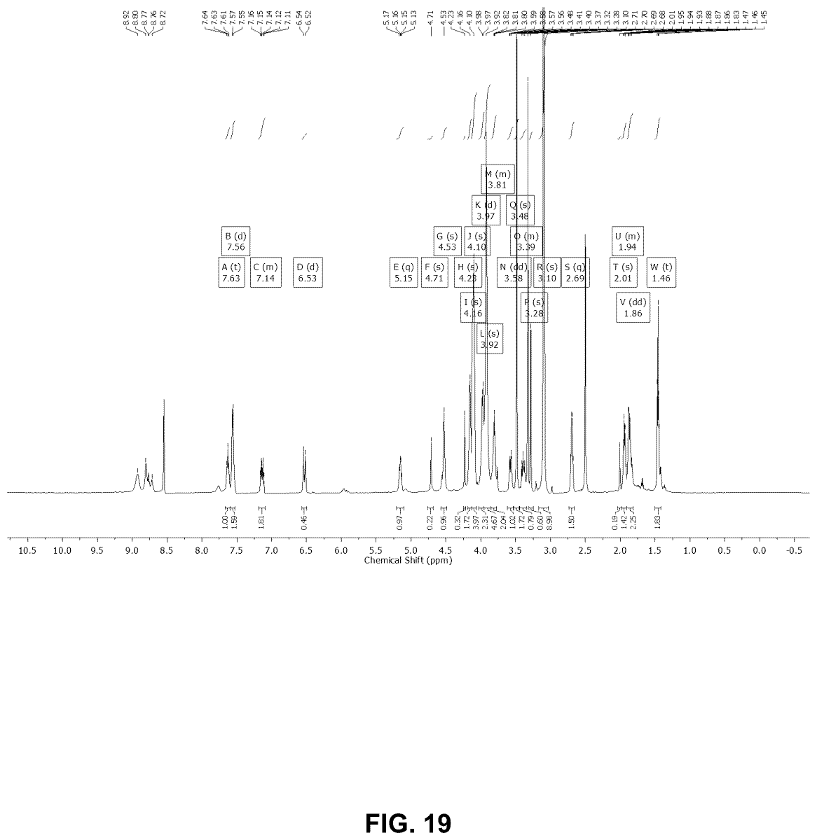

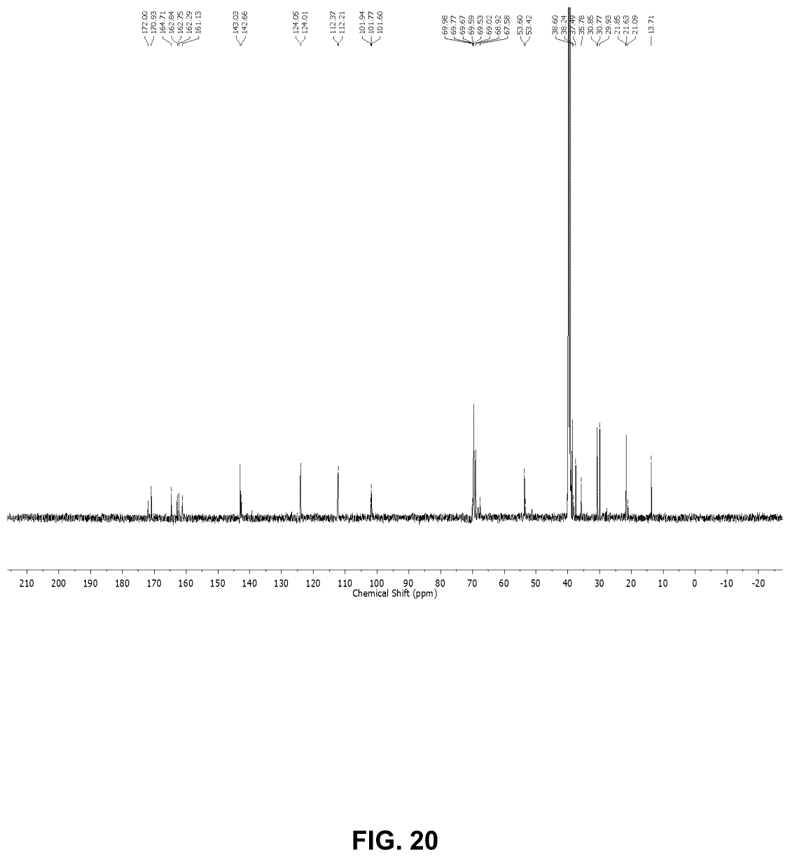

Hept-2-enoic acid (128.1 mg, 0.99 mmol) was dissolved in 2.0 mL anhydrous DMF and stirred for 5 min at room temperature with DIPEA (278.6 μL, 1.6 mmol) and HATU (380.1 mg, 0.99 mmol). Reaction transferred to vial containing 3. Reaction stirred 2.5 hours at room temperature then diluted with ethyl acetate and washed with 1 M HCl (3×15 mL) then dried over sodium sulfate. Solvent removed by rotary evaporation. The resulting colorless oil was purified by preparative TLC using 95:5 DCM:MeOH with 1% acetic acid (R_{f}=0.2). The product was extracted from the silica using ethyl acetate, and the solvent was removed by rotary evaporation, yielding 43.2 mg (0.09 mmol, 22%) of desired product PEG₃-4 (FIG. 18). 1H NMR (FIG. 19, FIG. 20) (600 MHz, DMSO-d6) δ 7.63 (t, J=9.0 Hz, 1H), 7.56 (d, J=6.8 Hz, 2H), 7.19-7.09 (m, 2H), 6.53 (d, J=15.4 Hz, OH), 5.15 (q, J=8.9 Hz, 1H), 4.71 (s, 1H), 4.53 (s, 1H), 4.23 (s, 1H), 4.16 (s, 2H), 4.10 (s, 4H), 3.97 (d, J=5.8 Hz, 2H), 3.92 (s, 5H), 3.85-3.77 (m, 2H), 3.58 (dd, J=4.0, 13.4 Hz, 1H), 3.48 (s, 2H), 3.44-3.34 (m, 1H), 3.28 (s, 1H), 3.10 (s, 9H), 2.69 (q, J=6.5 Hz, 1H), 2.01 (s, 1H), 1.98-1.91 (m, 1H), 1.86 (dd, J=10.9, 18.3 Hz, 2H), 1.46 (t, J=7.2 Hz, 2H). 13C NMR (151 MHz, DMSO-d6) δ 172.00, 170.93, 164.71, 162.84, 162.75, 162.29, 161.13, 143.03, 142.66, 124.05, 124.01, 112.37, 112.21, 101.94, 101.77, 101.60, 69.98, 69.77, 69.67, 69.59, 69.53, 69.02, 68.92, 67.58, 53.60, 53.42, 38.60, 38.24, 37.49, 35.78, 30.85, 30.77, 29.93, 21.85, 21.63, 21.09, 13.71. MS (ESI+): [M+H]⁺; found, 501.0

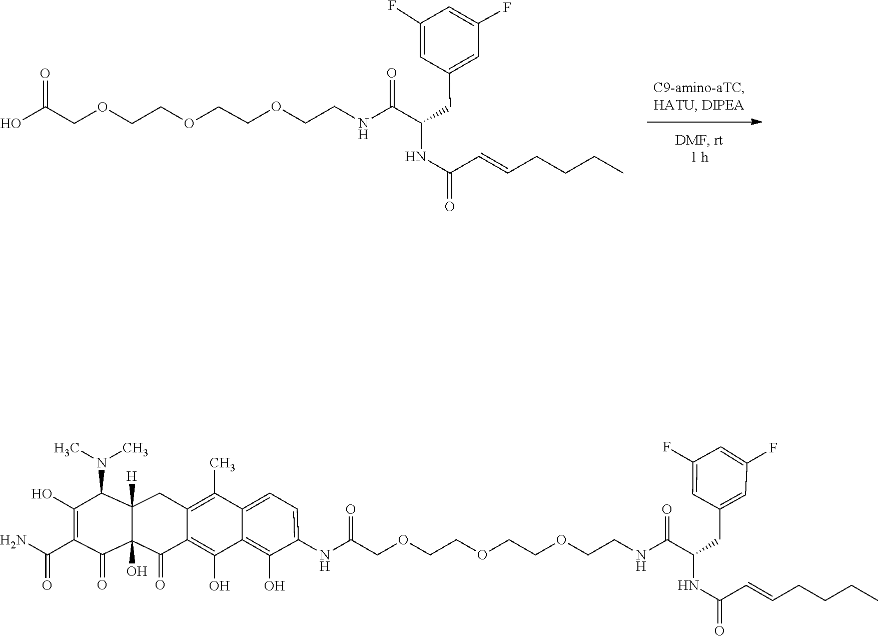 [SMILES: CCCC/C=C/C(=O)N[C@@H](Cc1cc(F)cc(F)c1)C(=O)NCCOCCOCCOCC(=O)O ; CCCC/C=C/C(=O)N[C@@H](Cc1cc(F)cc(F)c1)C(=O)NCCOCCOCCOCC(=O)Nc1ccc2c(C)c3c(c(O)c2c1O)C(=O)[C@]1(O)C(=O)C(C(N)=O)=C(O)[C@@H](N(C)C)[C@@H]1C3]

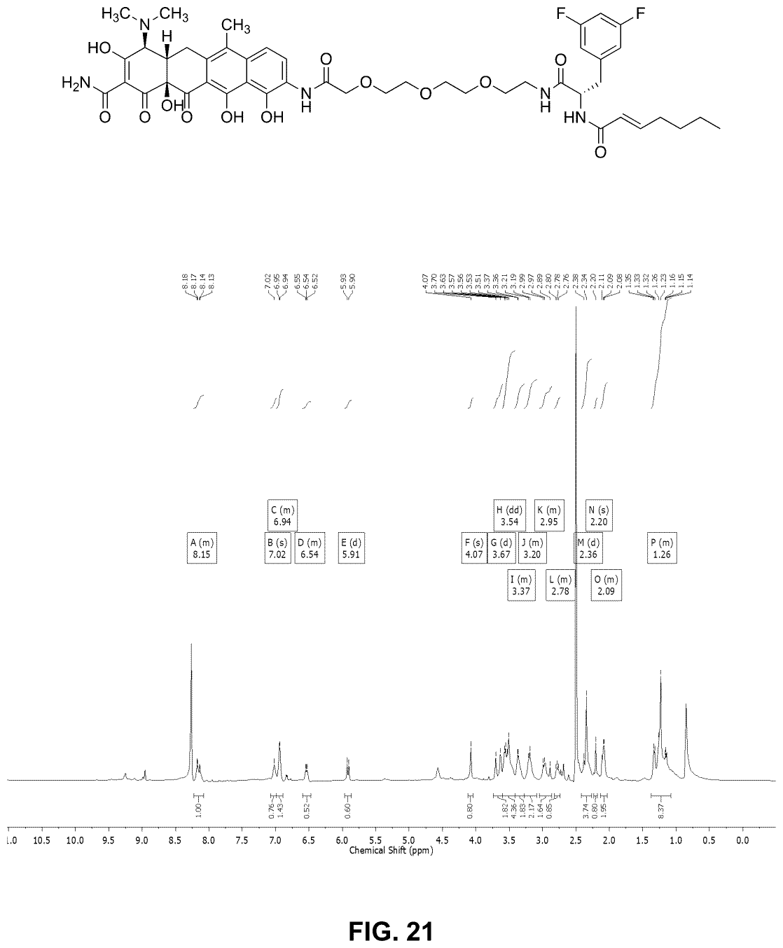

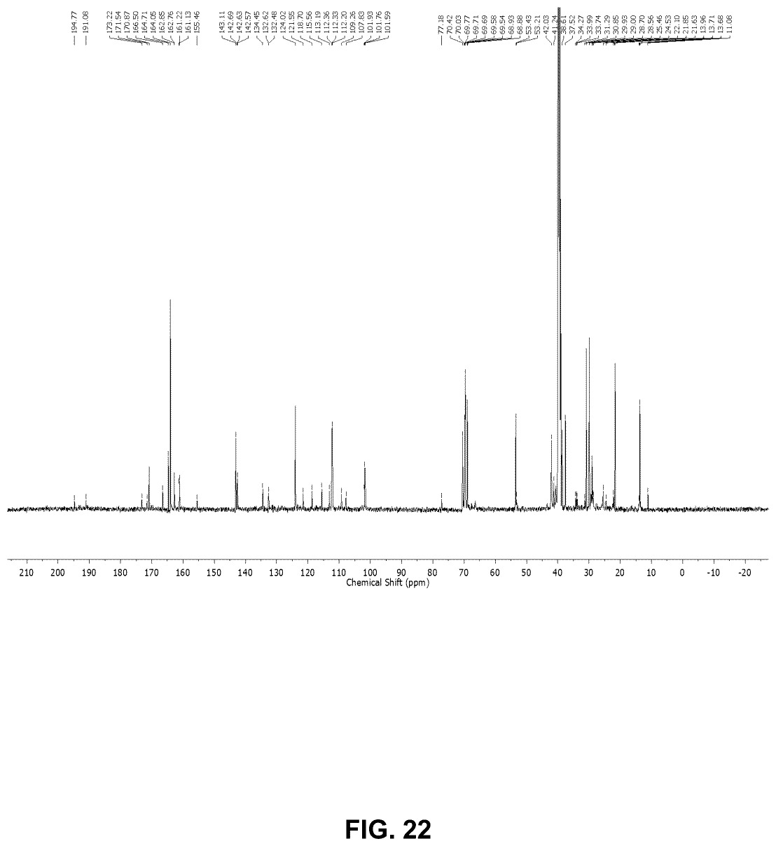

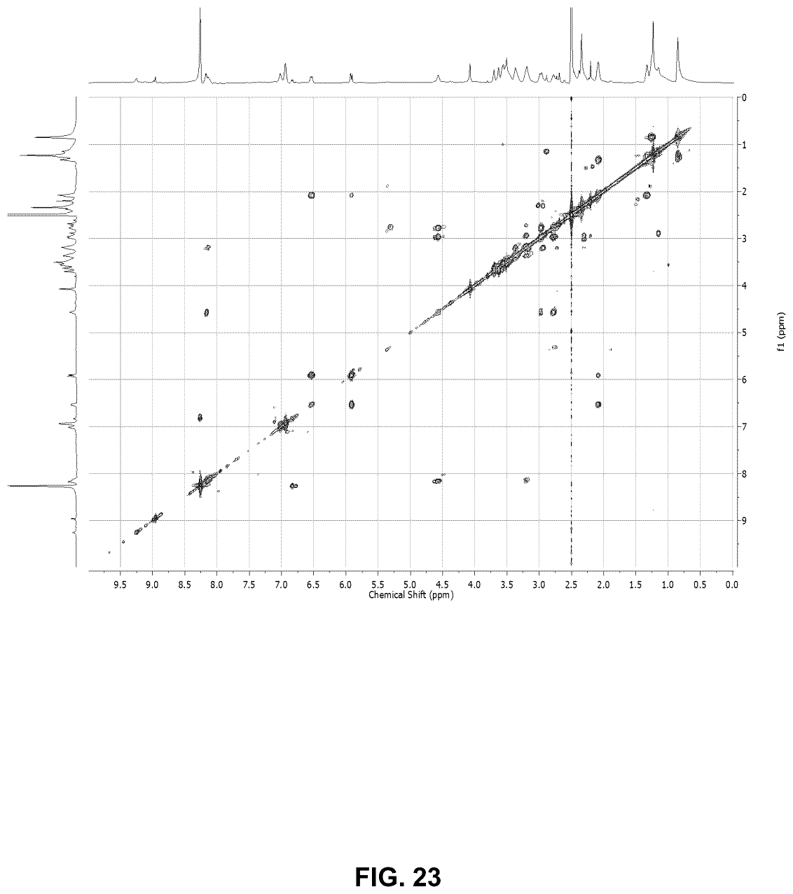

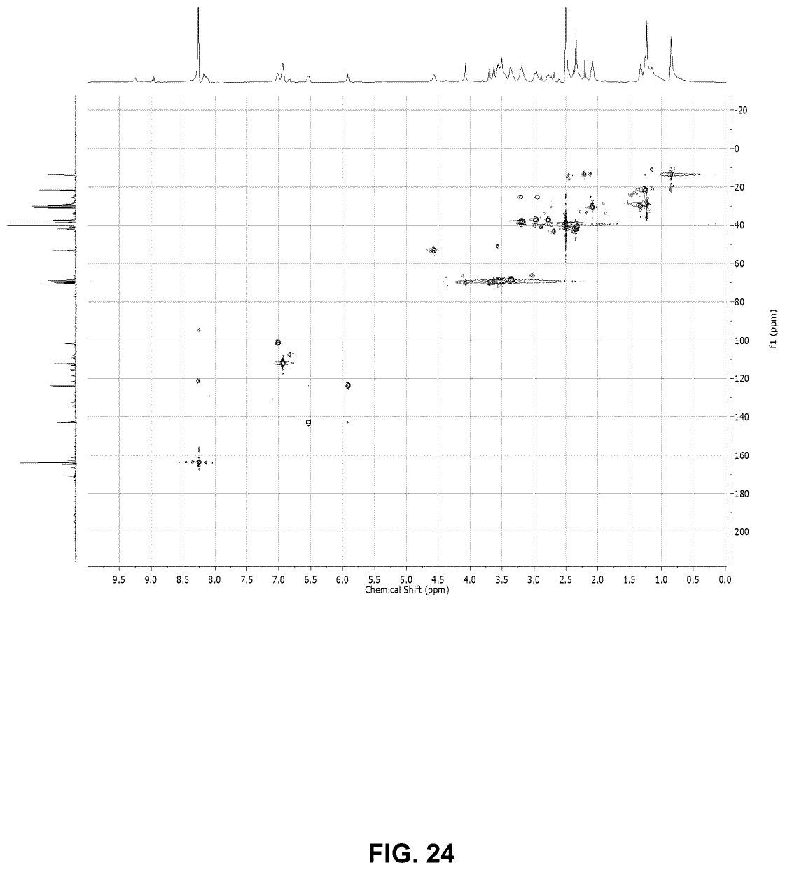

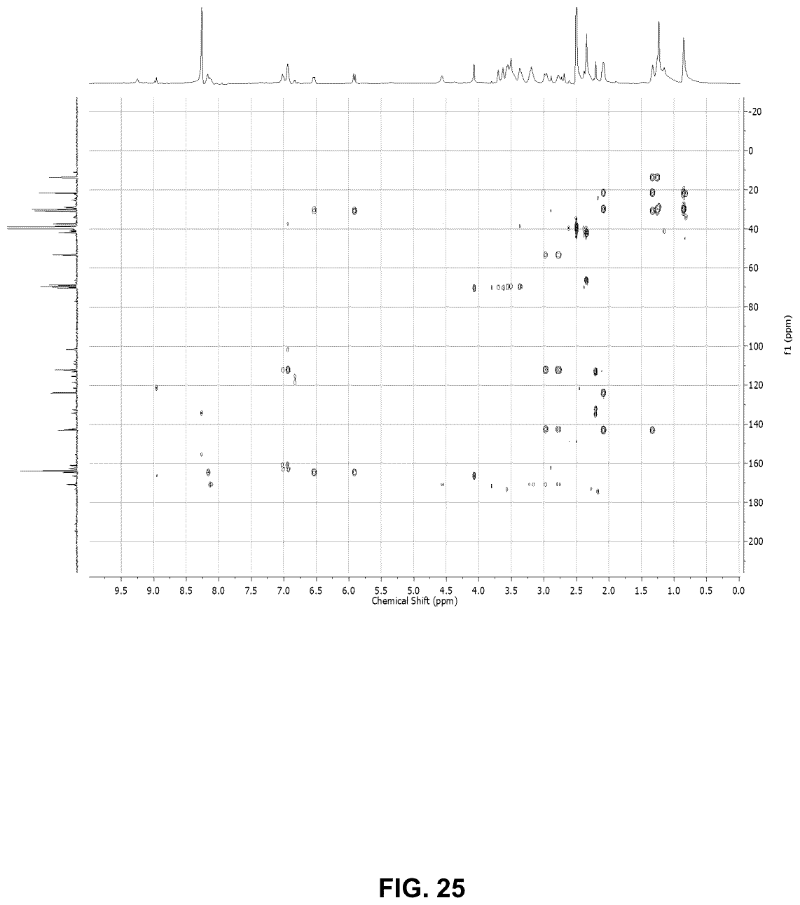

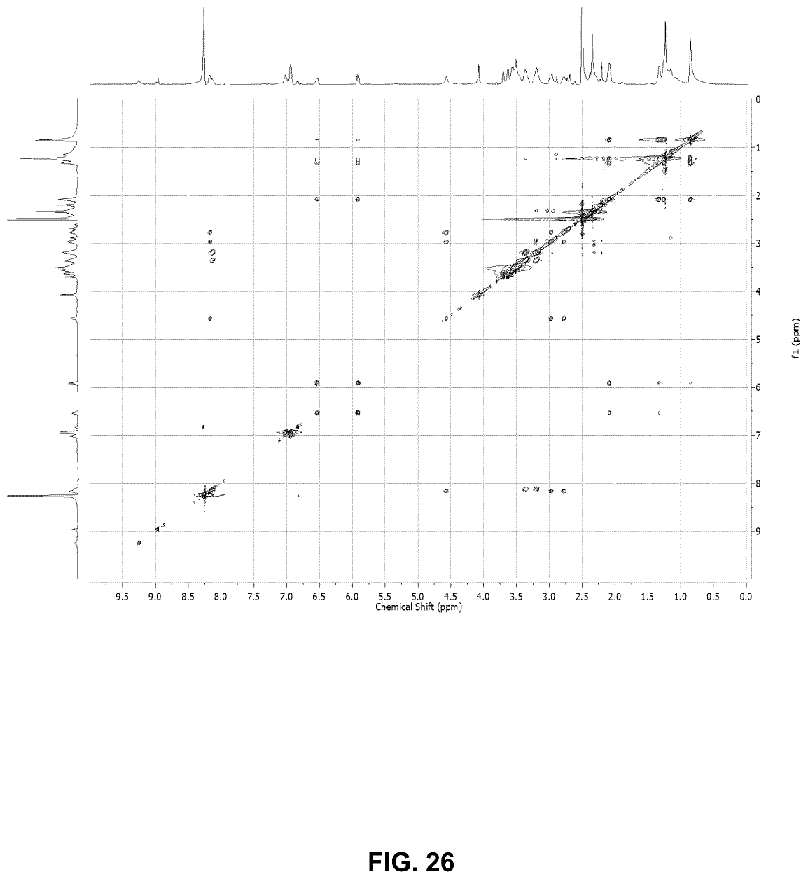

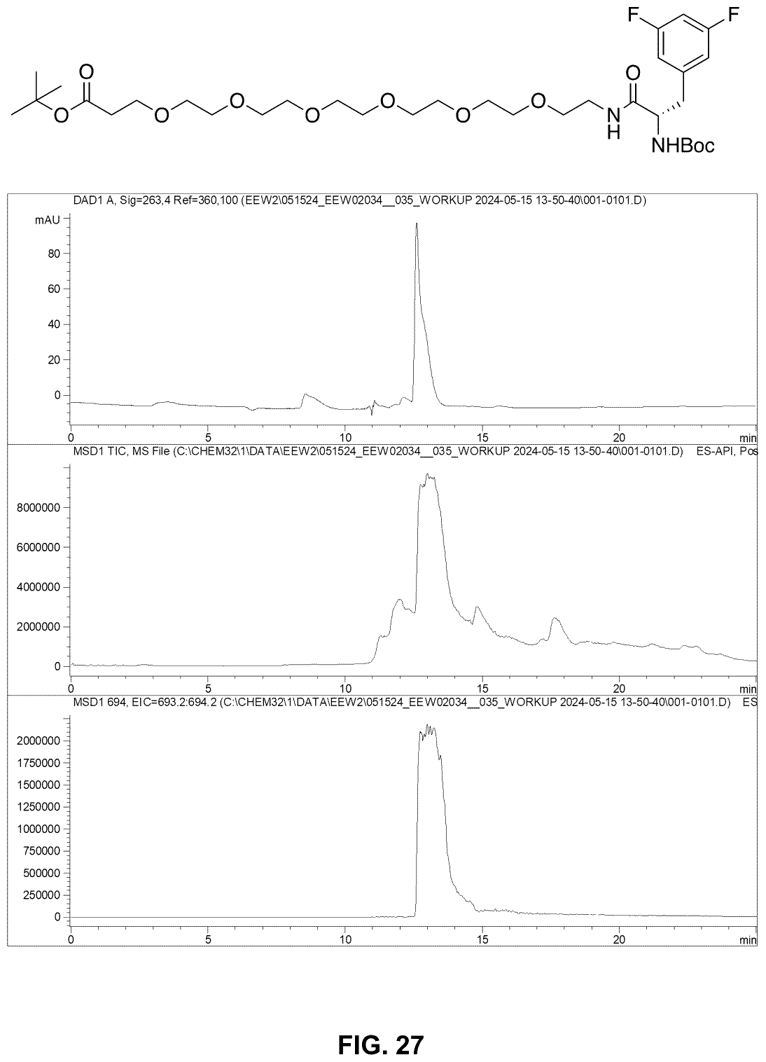

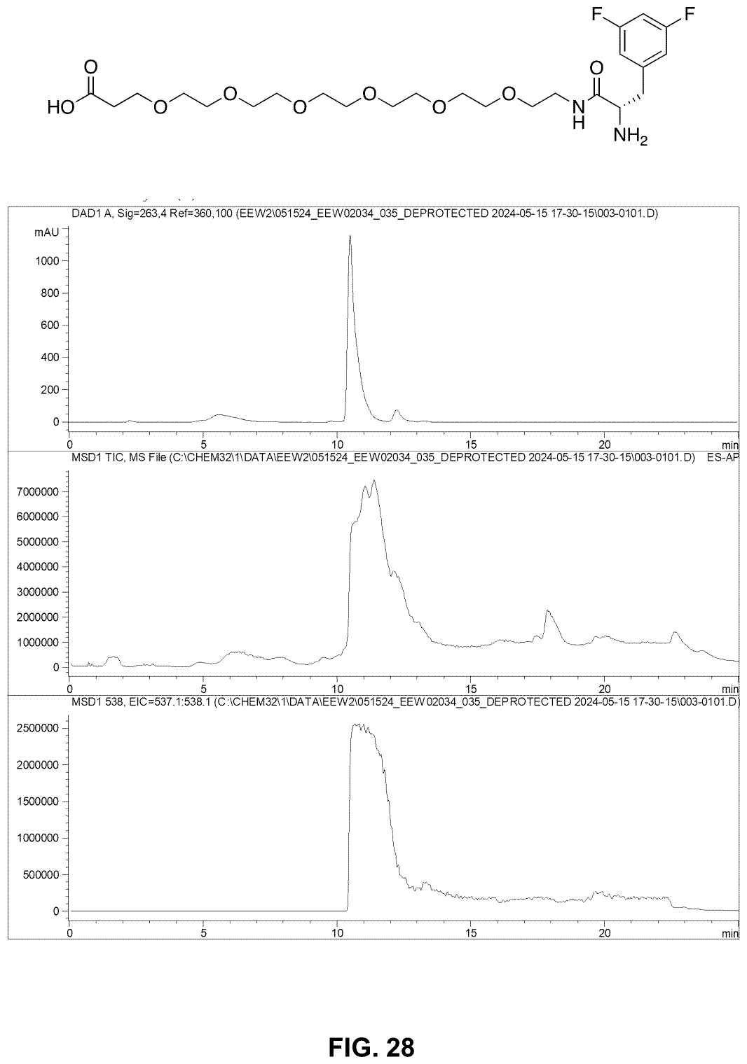

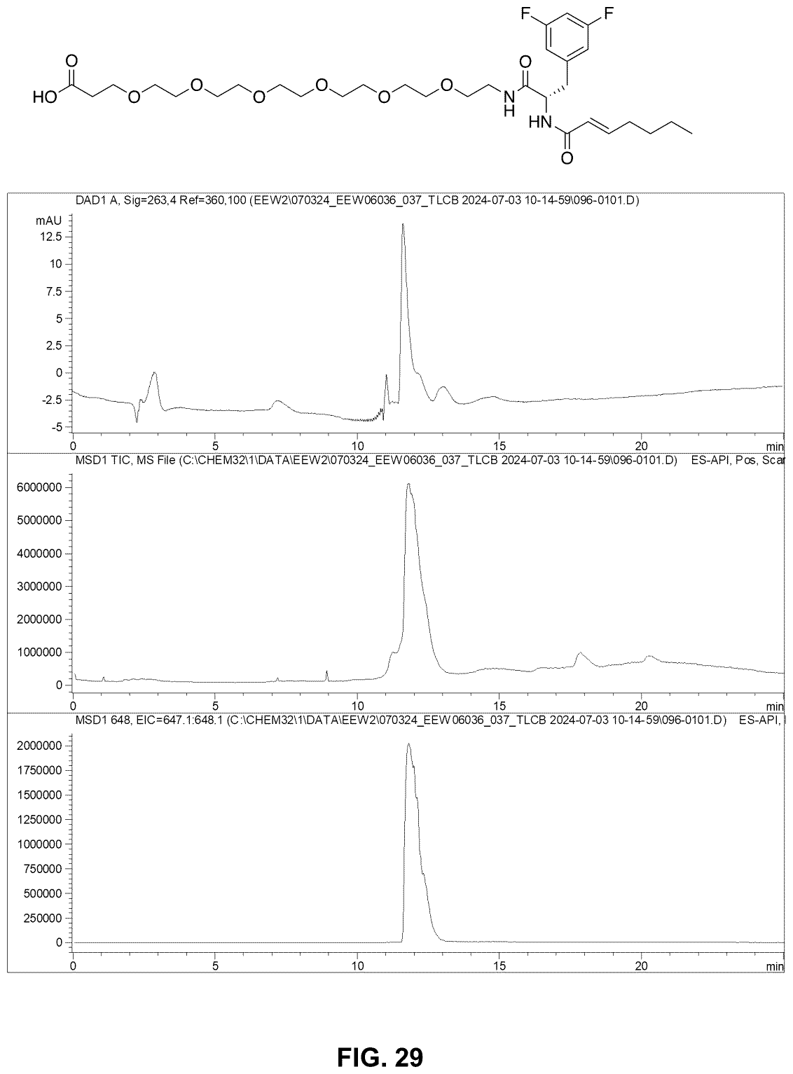

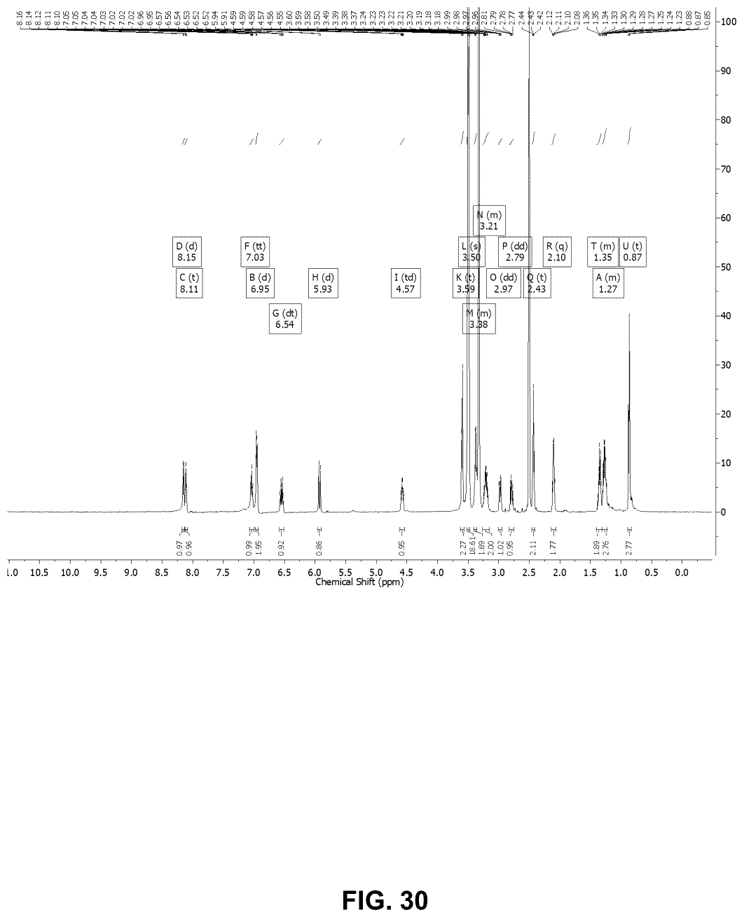

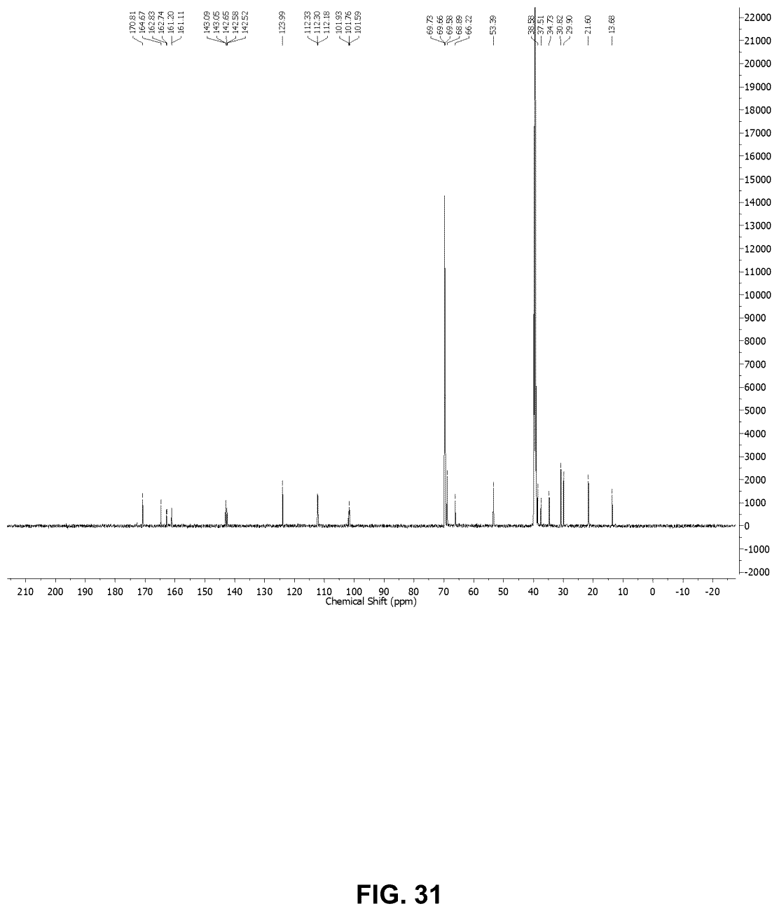

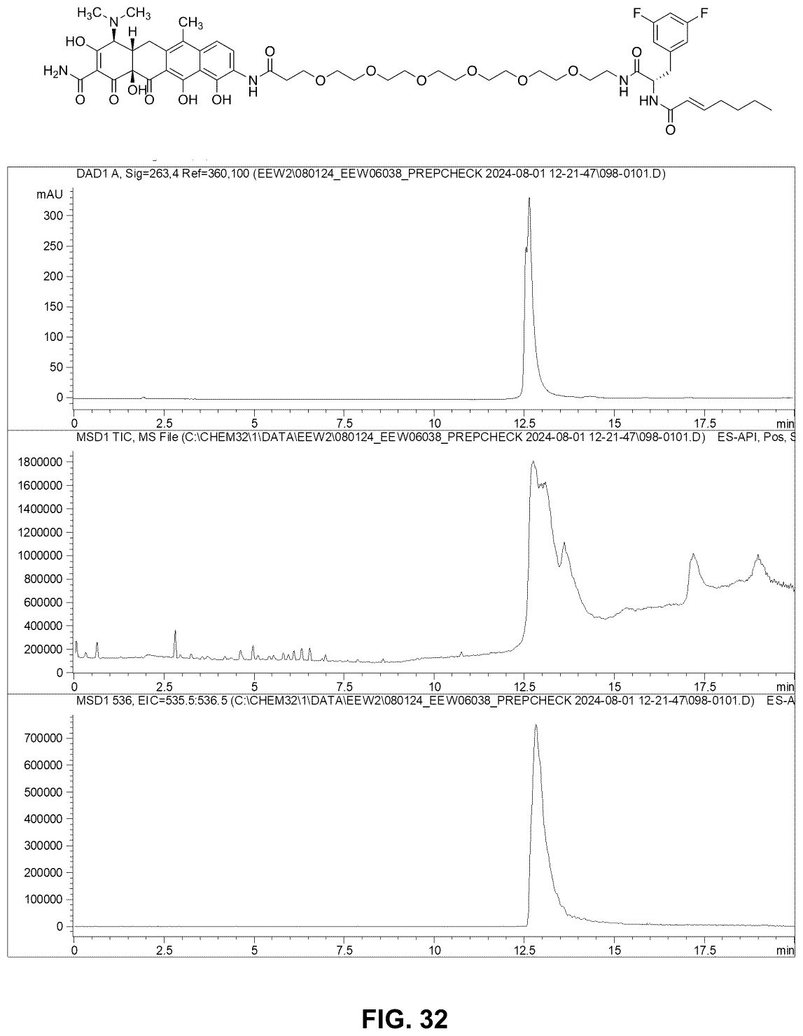

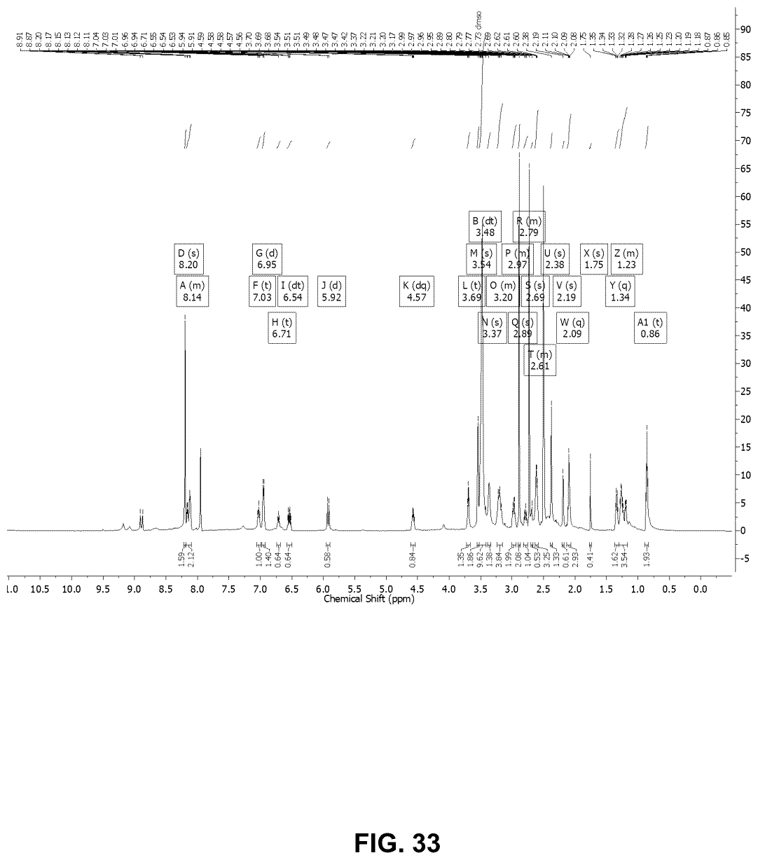

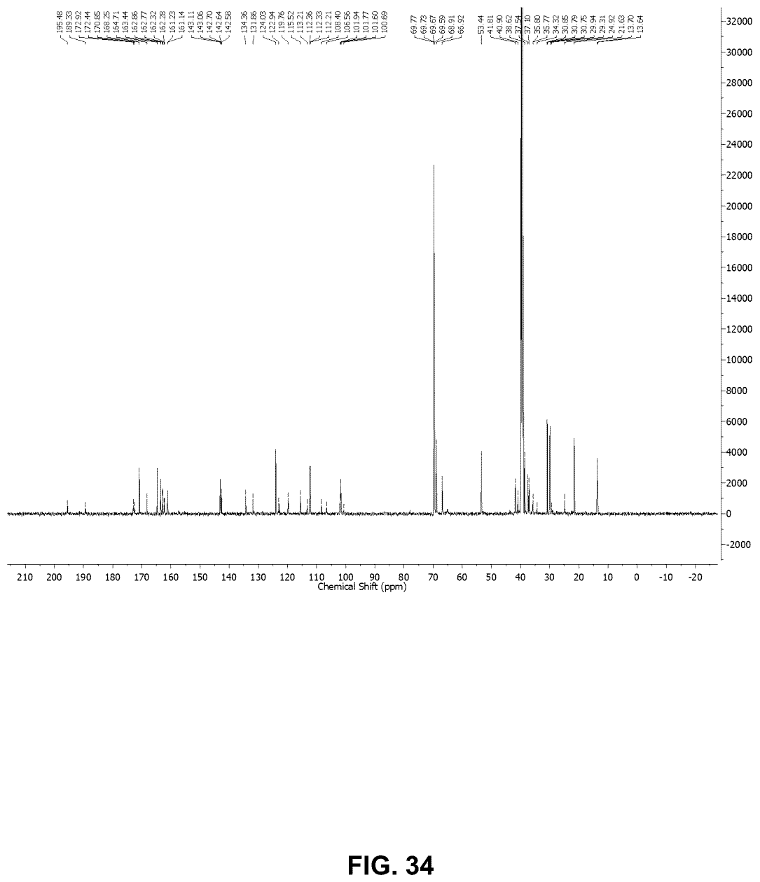

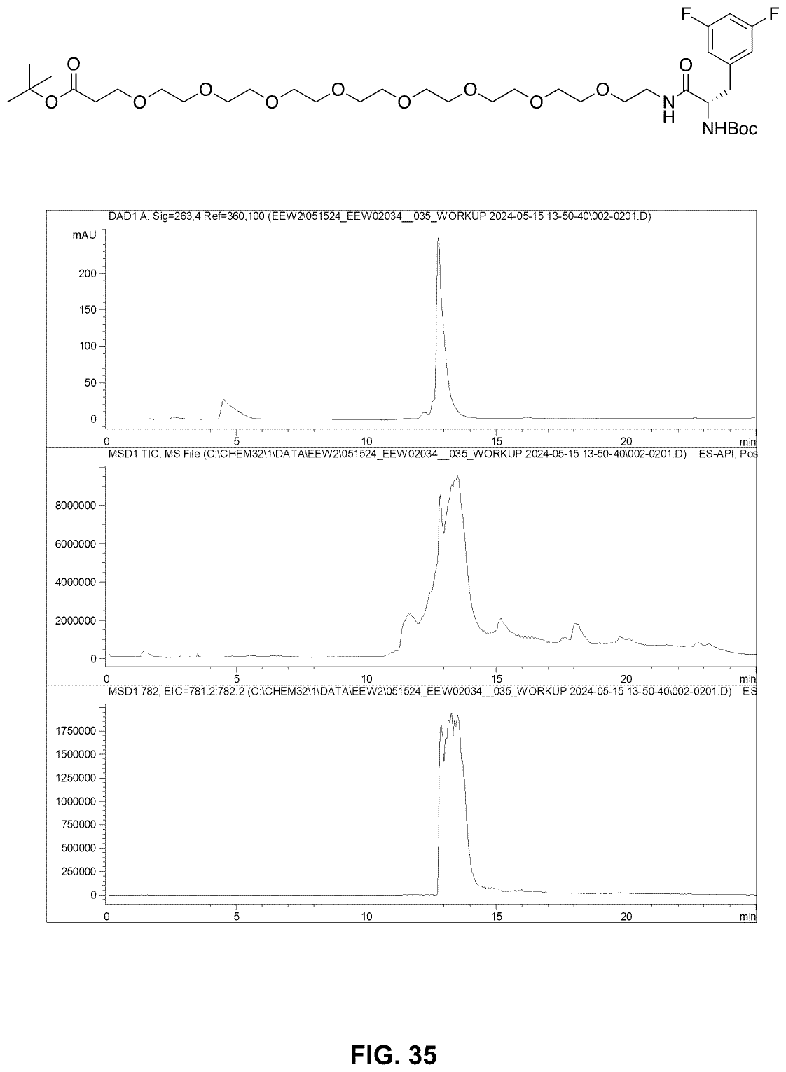

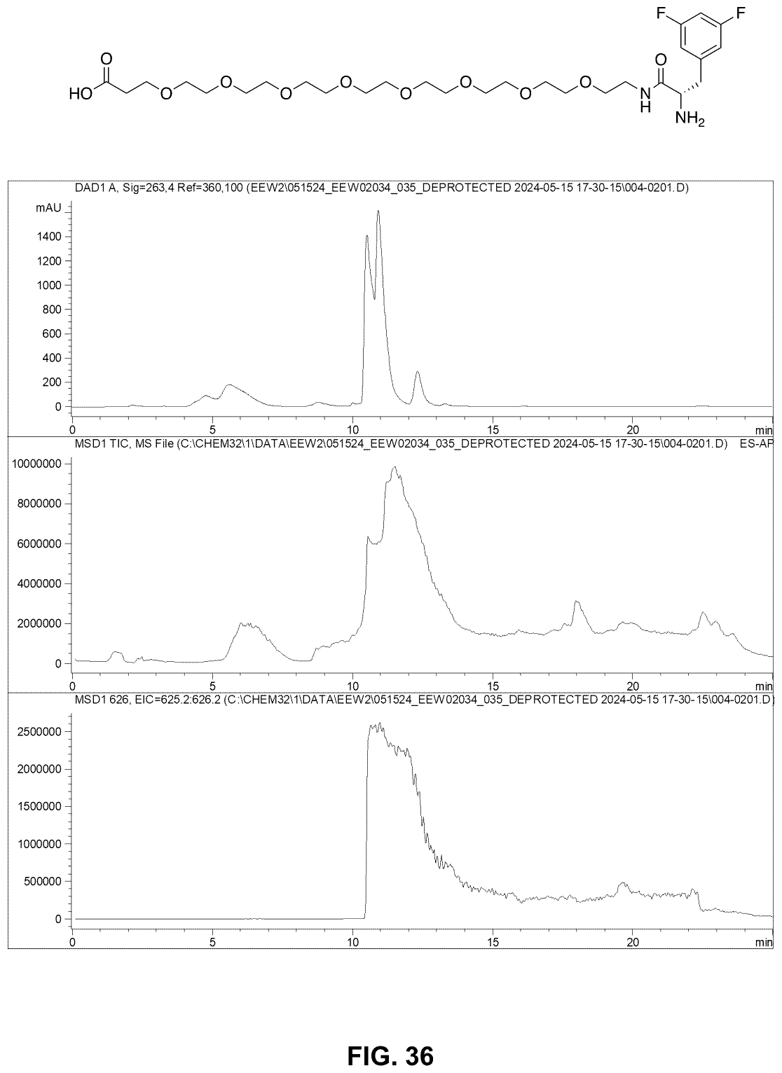

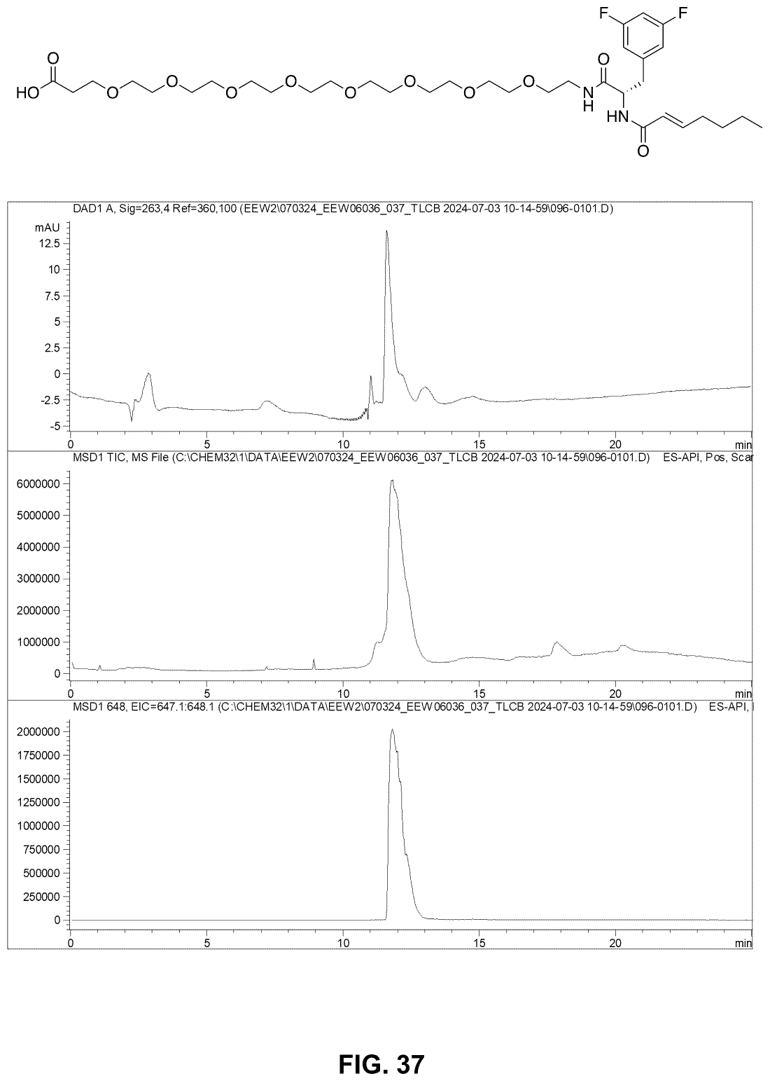

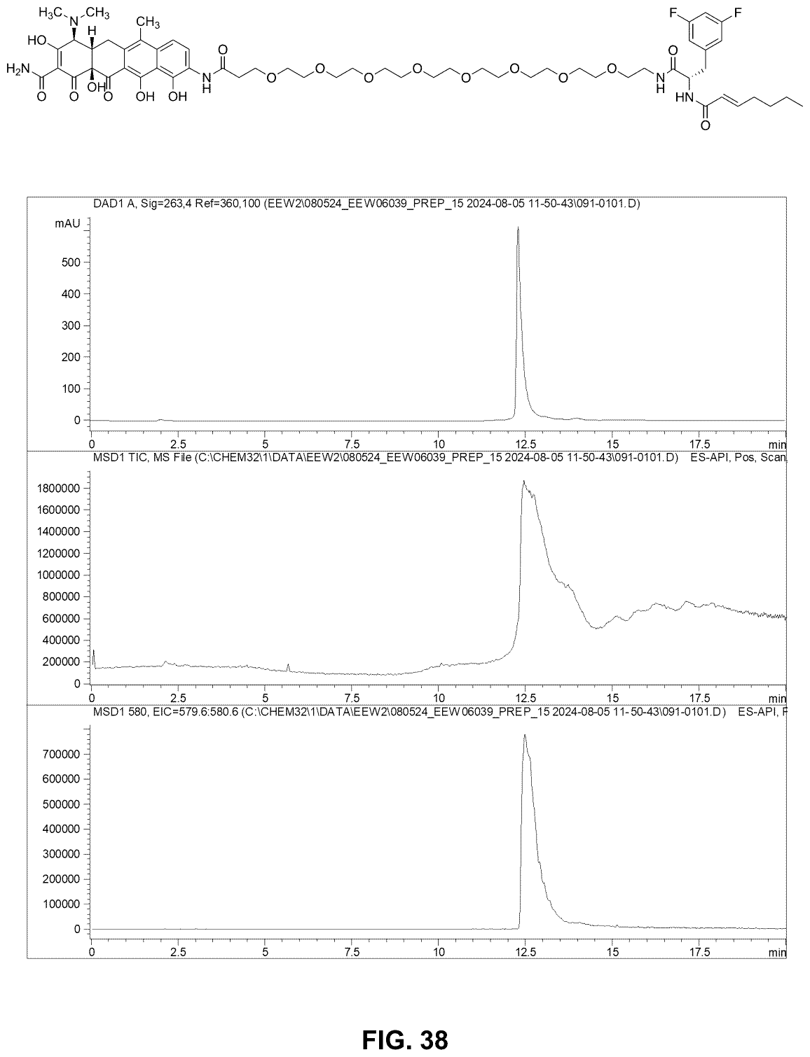

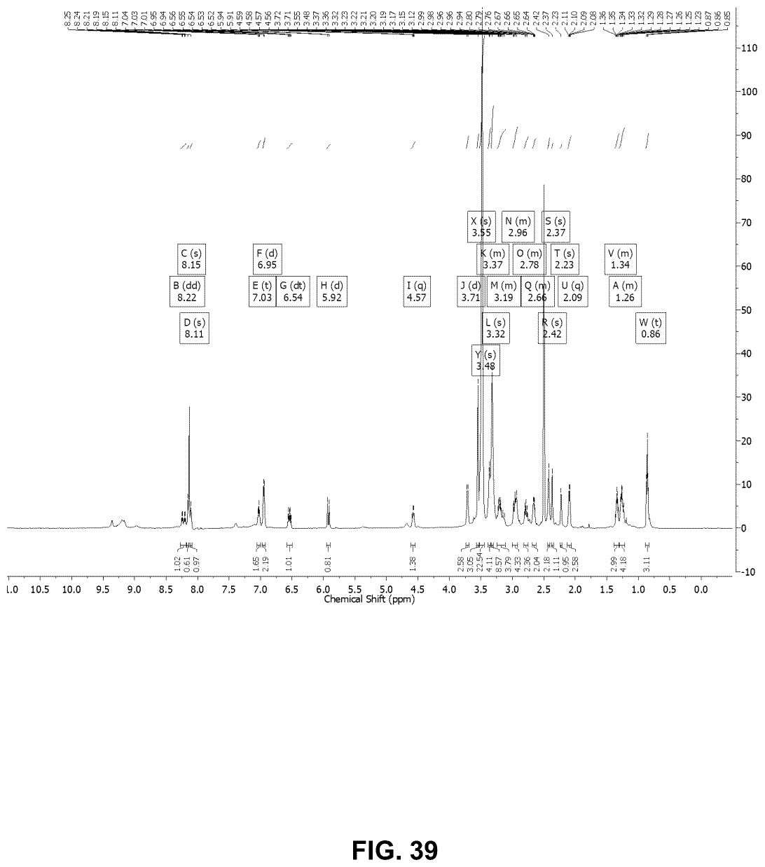

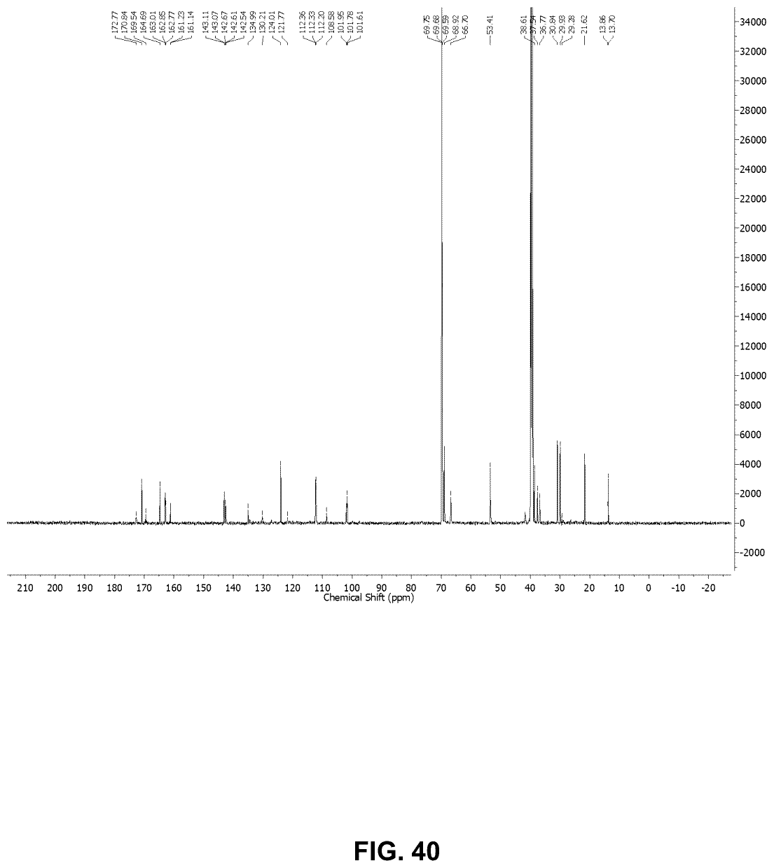

PEG₃-4 (43.2 mg, 0.09 mmol) was dissolved in 3.0 mL anhydrous DMF and stirred for 5 min at room temperature with DIPEA (37.3 μL, 0.21 mmol) and HATU (50.8 mg, 0.13 mmol). Reaction transferred to vial containing C9-amino-aTC (27.5 mg, 0.05 mmol). Reaction stirred at room temperature for 1 hour. 20 mL of ethyl acetate was added to reaction vial and cooled in fridge overnight at 4° C. to induce precipitation of desired product. Solvent was removed by pipette, remaining oil was dissolved in methanol, filtered through a 0.45 μm PTFE syringe filter, and purified by RP-C18 prep-HPLC to provide 16.5 mg (0.02 mmol, 22%) of the desired product PEG₃-5 as a dark red oil. 1H NMR (FIG. 21) (600 MHz, DMSO-d6) δ 8.22-8.08 (m, 1H), 7.02 (s, 1H), 6.99-6.89 (m, 1H), 6.59-6.47 (m, 1H), 5.91 (d, J=15.3 Hz, 1H), 4.07 (s, 1H), 3.67 (d, J=43.6 Hz, 2H), 3.54 (dd, J=12.4, 26.2 Hz, 4H), 3.42-3.27 (m, 2H), 3.27-3.09 (m, 2H), 3.05-2.86 (m, 2H), 2.83-2.74 (m, 1H), 2.36 (d, J=22.0 Hz, 4H), 2.20 (s, 1H), 2.13-2.03 (m, 2H), 1.37-1.07 (m, 8H). 13C NMR (FIG. 22) (151 MHz, DMSO-d6) δ 194.77, 191.08, 173.22, 171.54, 170.87, 166.50, 164.71, 164.05, 162.85, 162.76, 161.22, 161.13, 155.46, 143.11, 142.69, 142.63, 142.57, 134.45, 132.62, 132.48, 124.02, 121.55, 118.70, 115.56, 113.19, 112.36, 112.33, 112.20, 109.26, 107.83, 101.93, 101.76, 101.59, 77.18, 70.42, 70.03, 69.77, 69.71, 69.69, 69.58, 69.54, 68.93, 68.88, 53.43, 53.21, 42.03, 41.24, 38.61, 37.52, 34.27, 33.99, 33.74, 31.29, 30.85, 29.93, 29.00, 28.70, 28.56, 25.46, 24.53, 22.10, 21.85, 21.63, 13.96, 13.71, 13.68, 11.08. MS (ESI+): [M+H]⁺; found, 924.1

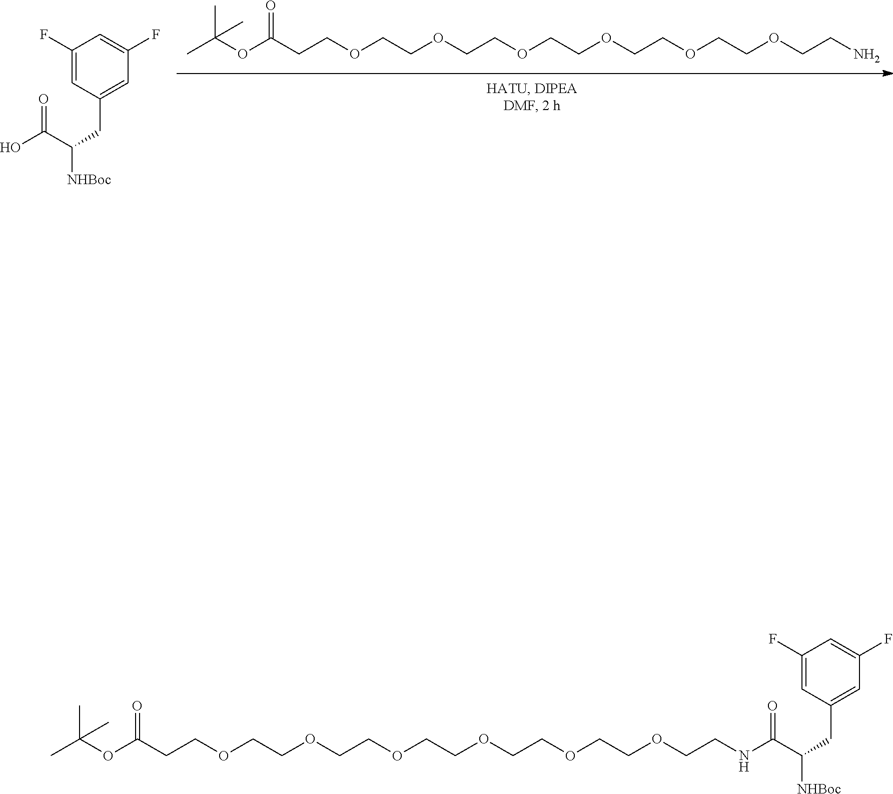

N-Boc-difluorophenylalanine (#, 200.7 mg, 0.67 mmol) was dissolved in 3.5 mL anhydrous DMF and stirred for 5 min at room temperature with DIPEA (105 μL, 0.61 mmol) and HATU (351.2 mg, 0.92 mmol). Reaction transferred to vial containing amino-PEG6-t-butyl-ester (247.9 mg, 0.61 mmol) and the colorless solution turned yellow. Reaction stirred for 2 hours at room temperature then diluted with ethyl acetate and washed with 1 M HCl (3×15 mL), followed by saturated sodium bicarbonate (2×15 mL). The organic layer was dried over sodium sulfate and the solvent was removed by rotary evaporation. The resulting yellow oil was purified by preparative TLC using 80:20 ethyl acetate:hexanes (R_{f}=0.4). The product was extracted from the silica using ethyl acetate, and the solvent was removed by rotary evaporation, yielding 290.9 mg (0.42 mmol, 68%) of desired product #. MS (ESI+): [M+H]⁺; found, 693.5

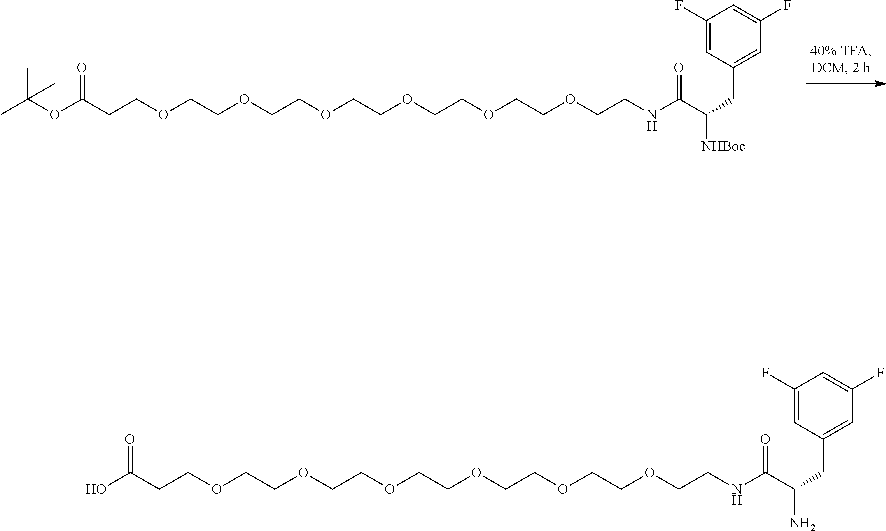 [SMILES: CC(C)(C)OC(=O)CCOCCOCCOCCOCCOCCOCCNC(=O)[C@H](Cc1cc(F)cc(F)c1)NC(=O)OC(C)(C)C ; N[C@@H](Cc1cc(F)cc(F)c1)C(=O)NCCOCCOCCOCCOCCOCCOCCC(=O)O]

Compound #(287.7 mg, 0.42 mmol) was dissolved in 1.2 mL DCM and 0.8 mL TFA and stirred at room temperature for 1.5 hours, yielding 274.8 mg (0.51 mmol, quant.) of desired product # as the corresponding TFA salt. H NMR 1H NMR (500 MHz, DMSO-d6) δ 7.17 (tt, J=2.3, 9.5 Hz, 1H), 6.97 (dd, J=2.1, 8.2 Hz, 1H), 4.01 (q, J=6.0, 6.6 Hz, 1H), 3.59 (d, J=12.7 Hz, 1H), 3.53-3.46 (m, 13H), 3.42 (d, J=3.8 Hz, OH), 3.42-3.30 (m, 2H), 3.25-3.14 (m, 1H), 3.09-3.02 (m, 1H), 2.98 (dd, J=7.9, 13.9 Hz, 1H), 2.89 (s, 1H), 2.73 (s, 1H), 2.43 (t, J=6.3 Hz, 1H). MS (ESI+): [M+H]⁺; found, 537.4

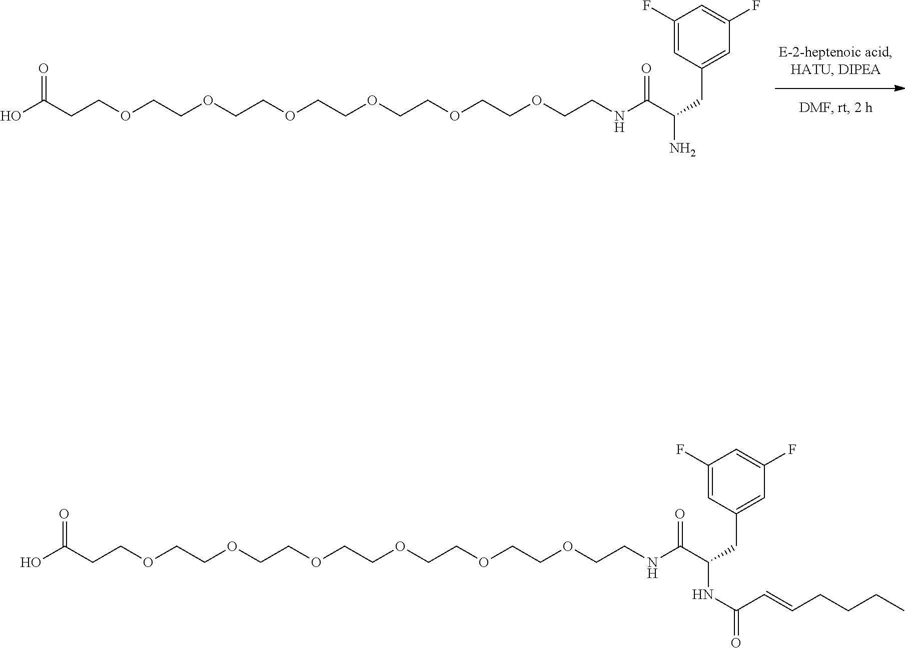 [SMILES: N[C@@H](Cc1cc(F)cc(F)c1)C(=O)NCCOCCOCCOCCOCCOCCOCCC(=O)O ; CCCC/C=C/C(=O)N[C@@H](Cc1cc(F)cc(F)c1)C(=O)NCCOCCOCCOCCOCCOCCOCCC(=O)O]

Hept-2-enoic acid (164.1 mg, 1.28 mmol) was dissolved in 2.0 mL anhydrous DMF and stirred for 5 min at room temperature with DIPEA (356.9 μL, 2.0 mmol) and HATU (486.7 mg, 1.3 mmol). Reaction transferred to vial containing #. Reaction stirred 2.5 hours at room temperature then diluted with ethyl acetate and washed with 1 M HCl (3×15 mL) then dried over sodium sulfate. Solvent removed by rotary evaporation. The resulting yellow oil was purified by preparative TLC using 95:5 DCM:MeOH with 1% acetic acid (R_{f}=0.2). The product was extracted from the silica using ethyl acetate, and the solvent was removed by rotary evaporation, yielding 73.8 mg (0.11 mmol, 22%) of desired product #. 1H NMR (600 MHz, DMSO-d6) δ 8.15 (d, J=8.6 Hz, 1H), 8.11 (t, J=5.7 Hz, 1H), 7.03 (tt, J=4.9, 7.0, 8.4 Hz, 1H), 6.95 (d, J=7.6 Hz, 2H), 6.54 (dt, J=7.1, 15.1 Hz, 1H), 5.93 (d, J=15.5 Hz, 1H), 4.57 (td, J=4.8, 9.2 Hz, 1H), 3.59 (t, J=6.3 Hz, 2H), 3.50 (s, 19H), 3.41-3.35 (m, 2H), 3.26-3.16 (m, 2H), 2.97 (dd, J=4.9, 13.7 Hz, 1H), 2.79 (dd, J=9.8, 13.8 Hz, 1H), 2.43 (t, J=6.3 Hz, 2H), 2.10 (q, J=7.1 Hz, 2H), 1.40-1.31 (m, 2H), 1.31-1.23 (m, 3H), 0.87 (t, J=7.2 Hz, 3H). 13C NMR (151 MHz, DMSO-d6) δ 170.81, 164.67, 162.83, 162.74, 161.20, 161.11, 143.09, 143.05, 142.65, 142.58, 142.52, 123.99, 112.33, 112.30, 112.18, 101.93, 101.76, 101.59, 69.73, 69.66, 69.58, 68.89, 66.22, 53.39, 38.58, 37.51, 34.73, 30.82, 29.90, 21.60, 13.68. MS (ESI+): [M+H]⁺; found, 647.4. HRMS (ESI+): [M+H]⁺; calc., 647.3350. Found, 647.3354.

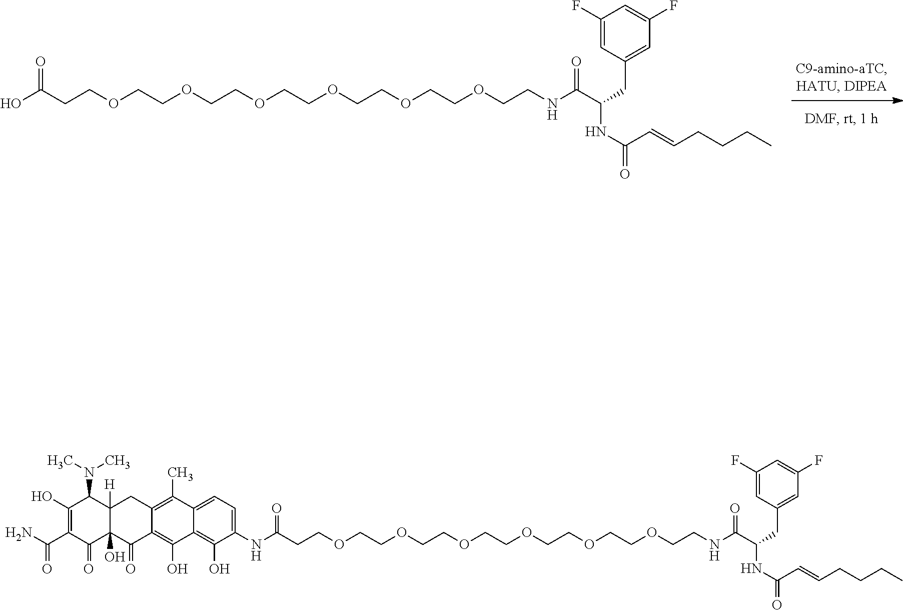 [SMILES: CCCC/C=C/C(=O)N[C@@H](Cc1cc(F)cc(F)c1)C(=O)NCCOCCOCCOCCOCCOCCOCCC(=O)O ; CCCC/C=C/C(=O)N[C@@H](Cc1cc(F)cc(F)c1)C(=O)NCCOCCOCCOCCOCCOCCOCCC(=O)Nc1ccc2c(C)c3c(c(O)c2c1O)C(=O)[C@]1(O)C(=O)C(C(N)=O)=C(O)[C@@H](N(C)C)C1C3]

#(73.8 mg, 0.11 mmol) was dissolved in 3.0 mL anhydrous DMF and stirred for 5 min at room temperature with DIPEA (49.6 μL, 0.28 mmol) and HATU (68 mg, 0.18 mmol). Reaction transferred to vial containing C9-amino-aTC (36.7 mg, 0.07 mmol). Reaction stirred at room temperature for 2 hour. 20 mL of ethyl acetate was added to reaction vial and cooled in fridge overnight at 4° C. to induce precipitation of desired product. Solvent was removed by pipette, remaining oil was dissolved in methanol, filtered through a 0.45 μm PTFE syringe filter, and purified by RP-C18 prep-HPLC to provide 10.4 mg (0.01 mmol, 14%) of the desired product PEG₃-5 as a dark red oil. 1H NMR (600 MHz, DMSO-d6) δ 8.20 (s, 2H), 8.18-8.09 (m, 2H), 7.03 (t, J=9.0 Hz, 1H), 6.95 (d, J=7.0 Hz, 1H), 6.71 (t, J=8.4 Hz, 1H), 6.54 (dt, J=6.9, 14.7 Hz, 1H), 5.92 (d, J=15.4 Hz, 1H), 4.57 (dq, J=4.4, 8.6 Hz, 1H), 3.69 (t, J=5.8 Hz, 1H), 3.54 (s, 2H), 3.48 (dt, J=6.4, 10.7 Hz, 10H), 3.37 (s, 1H), 3.24-3.15 (m, 4H), 3.00-2.93 (m, 2H), 2.89 (s, 2H), 2.82-2.75 (m, 1H), 2.69 (s, 1H), 2.64-2.59 (m, 3H), 2.38 (s, 1H), 2.19 (s, 1H), 2.09 (q, J=6.9, 7.4 Hz, 3H), 1.75 (s, OH), 1.34 (q, J=7.2 Hz, 2H), 1.30-1.17 (m, 4H), 0.86 (t, J=7.2 Hz, 2H). 13C NMR (151 MHz, dmso) δ 195.48, 189.33, 172.92, 172.44, 170.85, 168.25, 164.71, 163.44, 162.86, 162.77, 162.32, 162.28, 161.23, 161.14, 143.11, 143.06, 142.70, 142.64, 142.58, 134.36, 131.86, 124.03, 122.94, 119.76, 115.52, 113.21, 112.36, 112.33, 112.21, 108.40, 106.56, 101.94, 101.77, 101.60, 100.69, 69.77, 69.73, 69.67, 69.59, 68.91, 66.92, 53.44, 41.81, 40.90, 38.62, 37.54, 37.10, 35.80, 35.77, 34.32, 30.85, 30.79, 30.75, 29.94, 29.31, 24.92, 21.63, 13.70, 13.64. MS (ESI+): [M+2H]⁺; found, 535.8. HRMS (ESI+): [M+H]⁺; calc., 1070.4780. Found, 1070.4781.

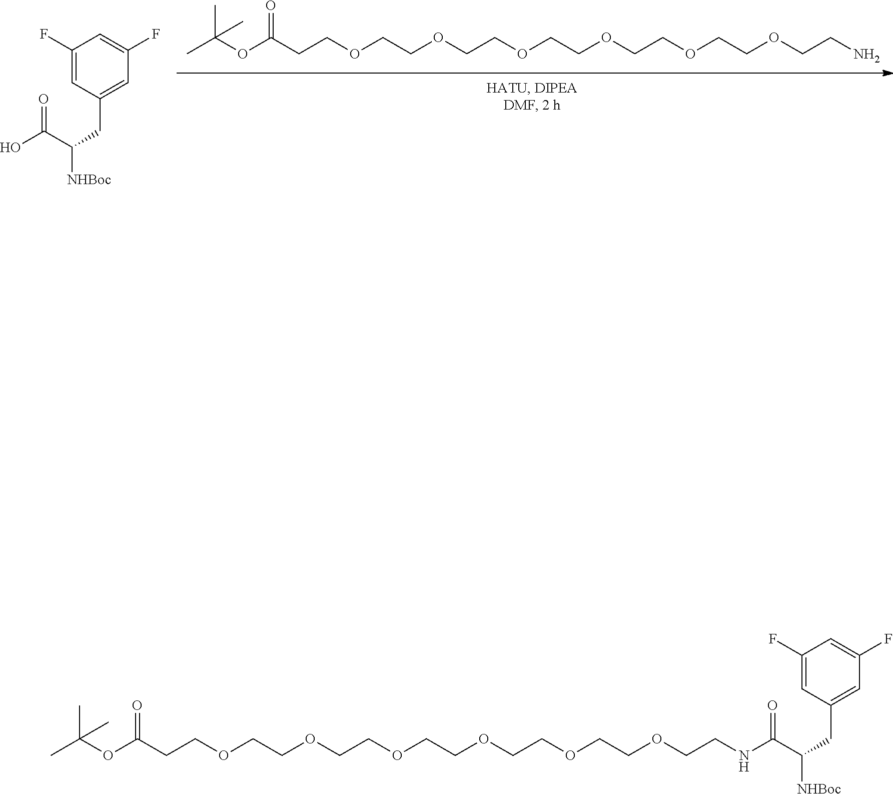

N-Boc-difluorophenylalanine (#, 204.8 mg, 0.68 mmol) was dissolved in 3.5 mL anhydrous DMF and stirred for 5 min at room temperature with DIPEA (108 μL, 0.62 mmol) and HATU (352.4 mg, 0.93 mmol). Reaction transferred to vial containing amino-PEG8-t-butyl-ester (307.5 mg, 0.62 mmol) and the colorless solution turned yellow. Reaction stirred for 2 hours at room temperature then diluted with ethyl acetate and washed with 1 M HCl (3×15 mL), followed by saturated sodium bicarbonate (2×15 mL). The organic layer was dried over sodium sulfate and the solvent was removed by rotary evaporation. The resulting yellow oil was purified by preparative TLC using 80:20 ethyl acetate:hexanes (R_{f}=0.4). The product was extracted from the silica using ethyl acetate, and the solvent was removed by rotary evaporation, yielding 226.4 mg (0.29 mmol, 47%) of desired product #. MS (ESI+): [M+H]⁺; found, 781.5

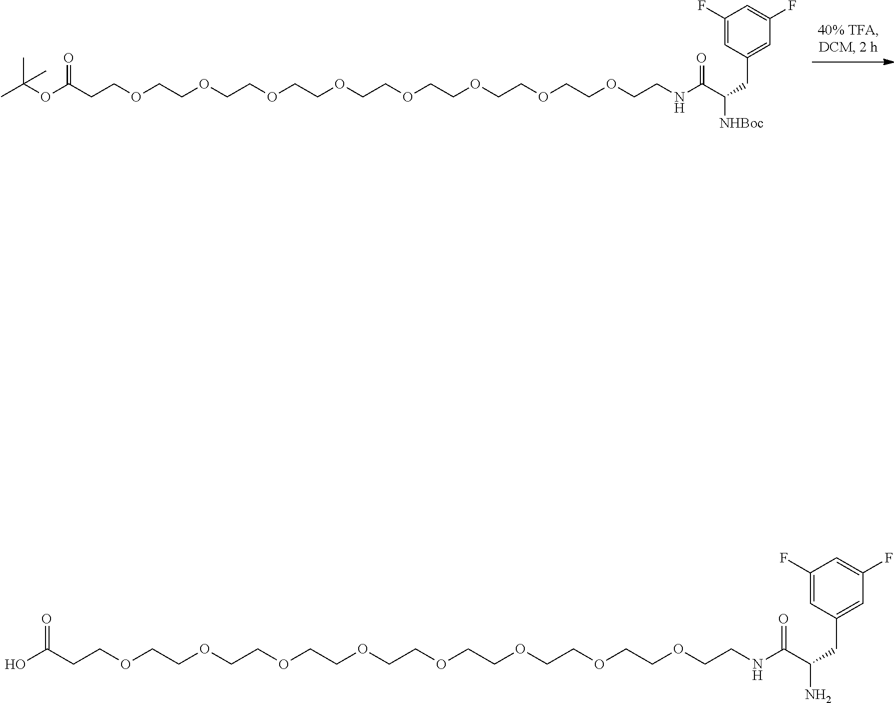 [SMILES: CC(C)(C)OC(=O)CCOCCOCCOCCOCCOCCOCCOCCOCCNC(=O)[C@H](Cc1cc(F)cc(F)c1)NC(=O)OC(C)(C)C ; N[C@@H](Cc1cc(F)cc(F)c1)C(=O)NCCOCCOCCOCCOCCOCCOCCOCCOCCC(=O)O]

Compound #(226.4 mg, 0.29 mmol) was dissolved in 1.2 mL DCM and 0.8 mL TFA and stirred at room temperature for 1.5 hours, yielding 183.0 mg (0.29 mmol, quant.) of desired product # as the corresponding TFA salt. H NMR 1H NMR (500 MHz, DMSO-d6) δ 7.21-7.14 (m, 1H), 6.97 (d, J=6.6 Hz, 2H), 4.05-3.98 (m, 1H), 3.62-3.56 (m, 9H), 3.42 (s, 1H), 3.39-3.31 (m, 3H), 3.23-3.16 (m, 1H), 3.06 (dd, J=6.4, 13.7 Hz, 2H), 3.02-2.94 (m, 1H), 2.89 (s, 8H), 2.73 (s, 7H), 2.69 (s, 14H), 2.44 (t, J=6.3 Hz, 9H). MS (ESI+): [M+H]⁺; found, 625.5

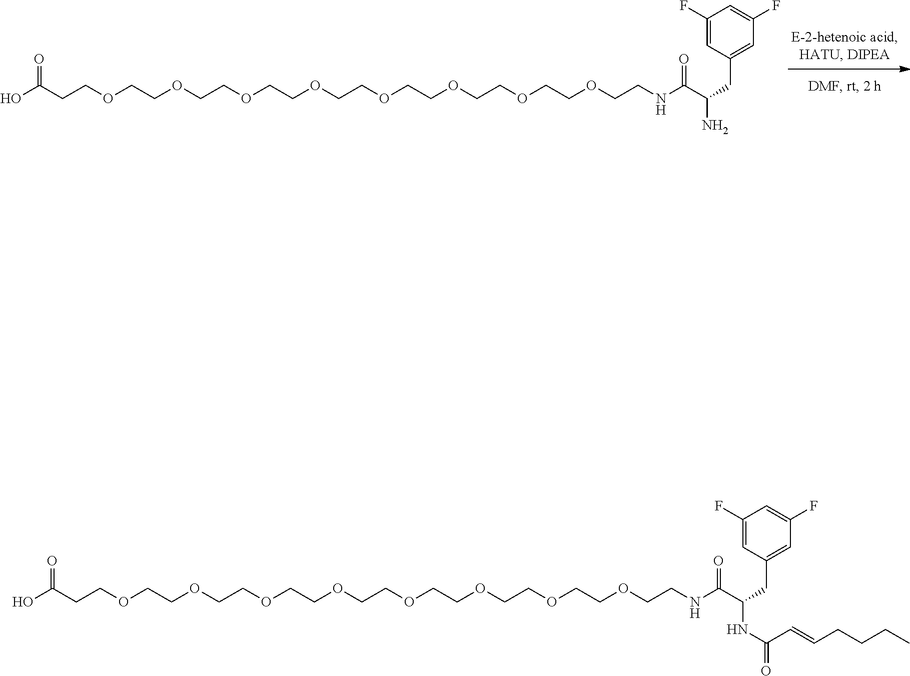 [SMILES: N[C@@H](Cc1cc(F)cc(F)c1)C(=O)NCCOCCOCCOCCOCCOCCOCCOCCOCCC(=O)O ; CCCC/C=C/C(=O)N[C@@H](Cc1cc(F)cc(F)c1)C(=O)NCCOCCOCCOCCOCCOCCOCCOCCOCCC(=O)O]

Hept-2-enoic acid (93.8 mg, 0.73 mmol) was dissolved in 2.5 mL anhydrous DMF and stirred for 5 min at room temperature with DIPEA (204.1 μL, 1.2 mmol) and HATU (278.5 mg, 0.73 mmol). Reaction transferred to vial containing #. Reaction stirred 2.5 hours at room temperature then diluted with ethyl acetate and washed with 1 M HCl (3×15 mL) then dried over sodium sulfate. Solvent removed by rotary evaporation. The resulting yellow oil was purified by preparative TLC using 95:5 DCM:MeOH with 1% acetic acid (R_{f}=0.2). The product was extracted from the silica using ethyl acetate, and the solvent was removed by rotary evaporation, yielding 49.7 mg (0.06 mmol, 21%) of desired product #. MS (ESI+): [M+H]⁺; found, 735.5. HRMS (ESI+): [M+H]⁺; calc., 735.3874. Found, 735.3885.

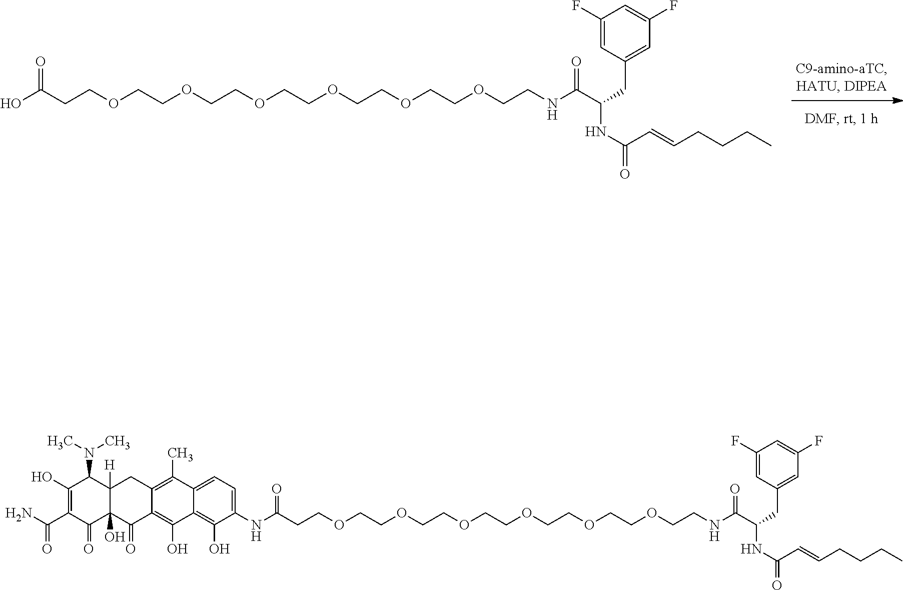 [SMILES: CCCC/C=C/C(=O)N[C@@H](Cc1cc(F)cc(F)c1)C(=O)NCCOCCOCCOCCOCCOCCOCCC(=O)O ; CCCC/C=C/C(=O)N[C@@H](Cc1cc(F)cc(F)c1)C(=O)NCCOCCOCCOCCOCCOCCOCCC(=O)Nc1ccc2c(C)c3c(c(O)c2c1O)C(=O)[C@]1(O)C(=O)C(C(N)=O)=C(O)[C@@H](N(C)C)C1C3]

#(44.2 mg, 0.06 mmol) was dissolved in 2.5 mL anhydrous DMF and stirred for 5 min at room temperature with DIPEA (26.2 PiL, 00.15 mmol) and HATU (35.7 mg, 0.09 mmol). Reaction transferred to vial containing C9-amino-aTC (19.3 mg, 0.04 mmol). Reaction stirred at room temperature for 2 hours. 20 mL of ethyl acetate was added to reaction vial and cooled in fridge overnight at 4° C. to induce precipitation of desired product. Solvent was removed by pipette, remaining oil was dissolved in methanol, filtered through a 0.45 μm PTFE syringe filter, and purified by RP-C18 prep-HPLC to provide 7.7 mg (0.01 mmol, 25%) of the desired product PEG₃-5 as a dark red oil. 1H NMR (600 MHz, DMSO-d6) δ 8.22 (dd, J=8.9, 25.3 Hz, 1H), 8.15 (s, 1H), 8.11 (s, 1H), 7.03 (t, J=8.7 Hz, 2H), 6.95 (d, J=7.0 Hz, 2H), 6.54 (dt, J=6.9, 14.5 Hz, 1H), 5.92 (d, J=15.4 Hz, 1H), 4.57 (q, J=8.8 Hz, 1H), 3.71 (d, J=5.7 Hz, 3H), 3.55 (s, 3H), 3.48 (s, 23H), 3.39-3.34 (m, 4H), 3.32 (s, 9H), 3.24-3.11 (m, 4H), 3.00-2.92 (m, 4H), 2.82-2.75 (m, 2H), 2.69-2.63 (m, 2H), 2.42 (s, 2H), 2.37 (s, 1H), 2.23 (s, 1H), 2.09 (q, J=6.5 Hz, 3H), 1.38-1.30 (m, 3H), 1.31-1.22 (m, 4H), 0.86 (t, J=7.1 Hz, 3H). 13C NMR (151 MHz, DMSO-d6) δ 172.77, 170.84, 169.54, 164.69, 163.01, 162.85, 162.77, 161.23, 161.14, 143.11, 143.07, 142.67, 142.61, 142.54, 134.99, 130.21, 124.01, 121.77, 112.36, 112.33, 112.20, 108.58, 101.95, 101.78, 101.61, 69.75, 69.68, 69.59, 68.92, 66.70, 53.41, 38.61, 37.54, 36.77, 30.84, 29.93, 29.28, 21.62, 13.86, 13.70. MS (ESI+): [M+2H]⁺; found, 579.9. HRMS (ESI+): [M+H]⁺; calc., 1158.5304. Found, 1158.5306.

What is claimed is:

1. A composition for degradation of a targeted enzyme, the composition comprising a bivalent agent, the bivalent agent comprising:
a. a targeted enzyme binding moiety configured to selectively bind to the targeted enzyme;
b. a proteasome targeting moiety configured to selectively bind to a peptidase component of a proteosome; and
c. a linker moiety, wherein the targeted protein binding moiety and protein degradation moiety are covalently linked at opposite ends of the linker moiety.

2. The composition of claim 1, wherein the proteasome targeting moiety comprises a peptide configured to selectively bind to a ClpP1P2 protease complex of the proteosome.

3. The composition of claim 2, wherein the proteasome targeting moiety is selected from a cyclic acyl depsipeptide (ADEP), a portion thereof, and (S,E)-(3,5 difluorophenyl-2-)hept-2-eneamido)propanoic acid (DFP).

4. The composition of claim 3, wherein the targeted enzyme binding moiety comprises a tetracycline destructase (TDase) inhibitor selected from anhydrotetracycline (aTC) or an aTC analog.

5. The composition of claim 4, wherein the linker molecule comprises a polyethylene glycol (PEG) linker.

6. The composition of claim 5, wherein the PEG linker comprises a linear series of PEGs ranging from about three PEGS (PEG₃) to about eight PEGS (PEG₈).

7. The composition of claim 6, wherein the bivalent agent comprises the aTC targeted enzyme binding moiety and the DFP proteasome targeting moiety covalently bound to opposite ends of the PEG linker.

8. The composition of claim 7, wherein the bivalent agent comprises a chemical structure according to Formula (I) below: 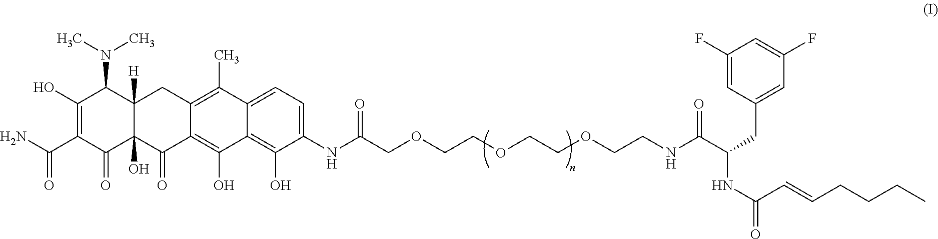
wherein n ranges from 3 to 8.

9. The composition of claim 8, wherein the bivalent agent is selected from aTC-PEG₃-DFP (PEG₃-5), aTC-PEG₃-DFP (PEG₆-5), and aTC-PEG₃-DFP (PEG₈-5) comprising the chemical structures illustrated below:

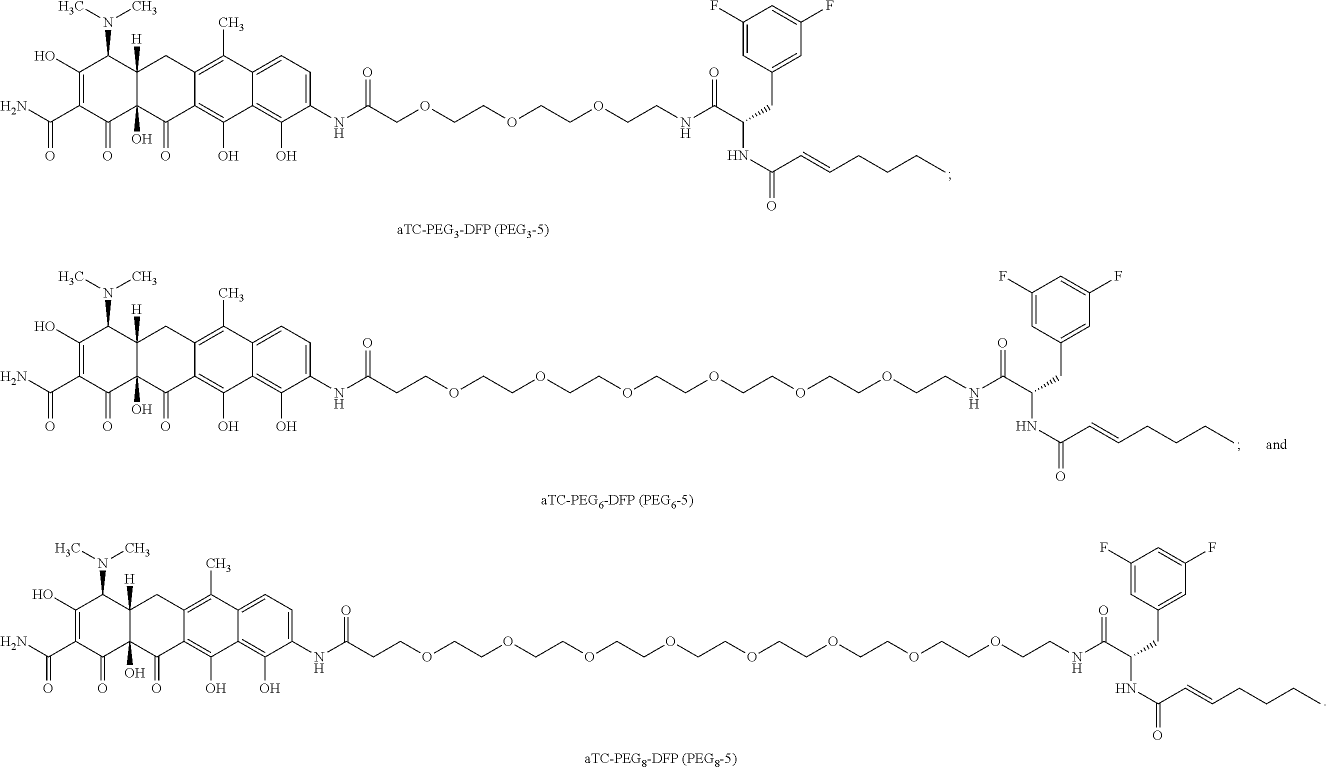 [SMILES: CCCC/C=C/C(=O)N[C@@H](Cc1cc(F)cc(F)c1)C(=O)NCCOCCOCCOCC(=O)Nc1ccc2c(C)c3c(c(O)c2c1O)C(=O)[C@]1(O)C(=O)C(C(N)=O)=C(O)[C@@H](N(C)C)[C@@H]1C3 ; CCCC/C=C/C(=O)N[C@@H](Cc1cc(F)cc(F)c1)C(=O)NCCOCCOCCOCCOCCOCCOCCC(=O)Nc1ccc2c(C)c3c(c(O)c2c1O)C(=O)[C@]1(O)C(=O)C(C(N)=O)=C(O)[C@@H](N(C)C)[C@@H]1C3 ; CCCC/C=C/C(=O)N[C@@H](Cc1cc(F)cc(F)c1)C(=O)NCCOCCOCCOCCOCCOCCOCCOCCOCCC(=O)Nc1ccc2c(C)c3c(c(O)c2c1O)C(=O)[C@]1(O)C(=O)C(C(N)=O)=C(O)[C@@H](N(C)C)[C@@H]1C3]

10. The composition of claim 8, wherein n is 6.

11. A method of reducing antibiotic resistance associated with an antibiotic-resistant bacterial infection in a subject, the method comprising administering a therapeutically effective amount of a composition comprising a bivalent agent to the subject, wherein the bivalent agent comprises a targeted enzyme binding moiety comprising anhydrotetracycline (aTC) and a proteasome targeting moiety comprising (S,E)-(3,5 difluorophenyl-2-)hept-2-eneamido)propanoic acid (DFP) attached at opposite ends of a polyethylene glycol (PEG) linker.

12. The method of claim 11, wherein the bivalent agent comprises a chemical structure according to Formula (I) below 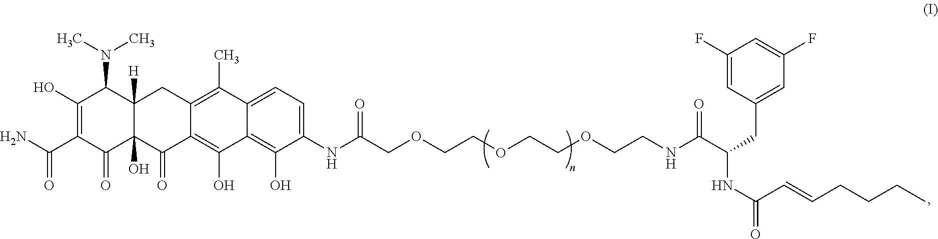
wherein n ranges from 3 to 8.

13. The method of claim 12, wherein the antibiotic-resistant bacterial infection is caused by a bacterial pathogen selected from Escherichia coli, Acinetobacter baumannii, Pseudomonas aeruginosa, Legionella longbeacha, and Mycobacteria abscessus.

14. The method of claim 13, wherein the composition administered to the subject comprises the bivalent agent at a concentration ranging from about 100 μM to about 1 mM.

15. A method of treating an antibiotic-resistant bacterial infection in a subject, the method comprising administering a therapeutically effective amount of a composition comprising a bivalent agent to the subject, wherein the bivalent agent comprises a targeted enzyme binding moiety comprising anhydrotetracycline (aTC) and a proteasome targeting moiety comprising (S,E)-(3,5 difluorophenyl-2-)hept-2-eneamido)propanoic acid (DFP) attached at opposite ends of a polyethylene glycol (PEG) linker.

16. The method of claim 15, wherein the bivalent agent comprises a chemical structure according to Formula (I) below 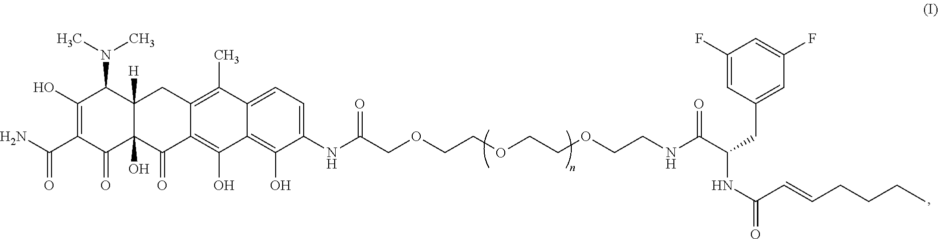
wherein n ranges from 3 to 8.

17. The method of claim 16, wherein the antibiotic-resistant bacterial infection is caused by a bacterial pathogen selected from Escherichia coli, Acinetobacter baumannii, Pseudomonas aeruginosa, Legionella longbeacha, and Mycobacteria abscessus.

18. The method of claim 17, wherein the composition administered to the subject comprises the bivalent agent at a concentration ranging from about 100 μM to about 1 mM.

19. The method of claim 18, wherein the composition administered to the subject further comprises a tetracycline antibiotic.

20. The method of claim 19, wherein the tetracycline antibiotic of the composition is administered to the subject at a concentration of about 8 μM.
